(19) United States
(12) Patent Application Publication
(10) Pub. No.: US 2020/0340011 A1
(43) Pub. Date: Oct. 29, 2020

(54) RECOMBINANT INSECT VECTORS AND METHODS OF USE

(71) Applicant: University of Wyoming, Laramie, WY (US)

(72) Inventors: Donald L. Jarvis, Laramie, WY (US); Hideaki Mabashi-Asazuma, Tokyo (JP)

(73) Assignee: University of Wyoming, Laramie, WY (US)

(21) Appl. No.: 16/308,076

(22) PCT Filed: Jun. 12, 2017
(86) PCT No.: PCT/US2017/037060

§ 371 Date: Dec. 7, 2018

Related U.S. Application Data
(60) Provisional application No. 62/348,674, filed on Jun. 10, 2016.

(51) Int. Cl. C12N 15/85; C12N 15/11

(52) U.S. Cl. CPC C12N 15/85; C12N 15/111; C12N 2710/14043; C12N 2330/51; C12N 2310/20

(57) ABSTRACT

The current teachings relate to DNA vectors for genomic editing in insect cells, for example glycoengineering in cell lines obtained from lepidopteran insects, and methods and kits for use of such vectors to modify genome editing function in insect cells. The disclosed vectors and methods comprise novel constructs that enable the CRISPR-Cas9 system in cultured insect cells. Also disclosed are lepidopteran cells that are transformed using the disclosed vectors and methods.

### CROSS-REFERENCE TO RELATED APPLICATIONS

This application claims the benefit of U.S. Provisional Application Ser. No. 62/348,674, filed Jun. 10, 2016, which is incorporated herein by reference in its entirety.

### STATEMENT REGARDING FEDERALLY SPONSORED RESEARCH

This work was performed in part with government support under Award Number R43 GM102982 from the National Institute of General Medical Sciences, National Institutes of Health. The Government may have certain rights in the claimed inventions.

### FIELD

The current teachings relate generally to the field of genomic editing. More particularly, the current teachings are directed to DNA vectors, methods, and kits for genomic editing and glycoengineering in insect cells, for example cell lines obtained from lepidopteran insects.

The baculovirus-insect cell system (BICS) has been widely used to produce many different recombinant proteins for basic research and is being used to produce several biologics approved for use in human or veterinary medicine. Early BICS were technically complex and constrained by the relatively primordial nature of insect cell protein glycosylation pathways. Since then, recombination has been used to modify baculovirus vectors, which has simplified the system, and to transform insect cells, which has enhanced its protein glycosylation capabilities.

CRISPR-Cas9 is a powerful site-specific genome-editing tool that has been used to genetically engineer many different systems. CRISPR-Cas9 tools for site-specific genome editing are needed to facilitate further improvements in the BICS

### BACKGROUND

The BICS, first described in 1983, has been used to produce thousands of different recombinant proteins for diverse areas of biomedical research. Since 2009, the BICS also has been used to produce several biologics approved for use in human or veterinary medicine. Thus, the BICS is an important recombinant protein production platform that has had and will continue to have a large and broad impact on basic research, biotechnology, and medicine.

Two precedents suggest the BICS would have even more impact if it could be engineered to enhance its capabilities and/or extend its utility. In the 1980's, the isolation of baculovirus expression vectors was a highly inefficient, time-consuming, and frustrating process. However, by the early 1990's, efforts to engineer the baculoviral genome in various ways had greatly simplified this process. These refinements effectively converted a complex system created in highly specialized labs to a routine tool that could be easily used in many different labs. This was followed by efforts to enhance the BICS by engineering host protein N-glycosylation pathways. However, host glycoengineering and other host improvement efforts have been limited to the use of non-homologous recombination to knock-in heterologous genes at random sites. This is because there have been no tools for site-specific genome manipulation in the insect cell lines most commonly used as hosts in the BICS. These cell lines include Sf9 and HIGH FIVE™, which are derived from the lepidopteran insects, Spodoptera frugiperda (Sf) and Trichoplusia ni (Tn), respectively.

Sf9 and HIGH FIVE® cells have the machinery required for protein N-glycosylation, but cannot synthesize the same end products as mammalian cells. More specifically, insect and mammalian cells can both transfer N-glycan precursors to nascent polypeptides and trim those precursors to produce identical processing intermediates. However, insect cells lack the additional machinery needed to elongate those intermediates and produce larger, mammalian-like structures with new terminal sugars, such as sialic acids. Insect cells also have a trimming enzyme, absent in mammalian cells, which antagonizes N-glycan elongation. This enzyme, which is a specific, processing ß-N-acetylglucosaminidase called fused lobes (FDL), removes a terminal N-acetylglucosamine residue from trimmed N-glycan processing intermediates. This antagonizes elongation because it eliminates the N-glycan intermediates used as substrates for N-acetylglucosaminyltransferase II, which initiates the elongation process. The inability of the BICS to produce mammalian-type, elongated N-glycans is a major deficiency of this system because these structures are required for clinical efficacy in glycoprotein biologics. Due to its inability to synthesize these structures, it is widely believed that the BICS platform could never be used for glycoprotein biologics manufacturing.

This limitation has been addressed by using non-homologous recombination to engineer insect cell N-glycosylation pathways for mammalian-type N-glycan biosynthesis. These efforts have yielded new, transgenic insect cell lines that can be used to produce recombinant glycoproteins with fully elongated, mammalian-type N-glycans. However, further glycoengineering is needed to create host cell lines that can more efficiently process N-glycans in mammalian fashion and produce homogeneously glycosylated proteins. These more refined glycoengineering efforts will require tools for site-specific genome editing in the BICS and fdl, which encodes an antagonistic function, will be a critically important target.

For at least the foregoing reasons, there is a need for tools to allow site-specific genome editing in insect cell lines, particularly cell lines used to produce recombinant proteins and biologics for human and veterinary uses. There is also a need for recombinant vectors that are capable of altering protein glycosylation pathways in insect cells, for example, the BICS.

### SUMMARY

The disclosed teachings provide DNA vectors and methods for using the disclosed vectors for site-specific genome editing in insect cells, for example but not limited to, cultured Sf and Tn cells such as the Sf9, Sf21, Sf-RVN, Tn-368, EXPRESSF+®, SUPER 9®, HIGH FIVE®^{M}, and TNI PRO® cell lines.

According to certain embodiments, DNA vectors comprise: a Streptococcus pyogenes Cas9 (SpCas9) coding sequence operably linked to a first transcription control element; a single guide RNA (sgRNA) expression cassette comprising a targeting sequence cloning site and a sgRNA coding sequence operably linked to a second transcription control element; and a selectable marker operably linked to a third transcription control element.

Certain method embodiments for obtaining a modified lepidopteran cell comprising a newly introduced genome editing function resulting in a modified cellular phenotype comprise transfecting a lepidopteran insect cell with a DNA vector of the current teachings, wherein the vector comprises a targeting sequence inserted into the target sequence cloning site and operably linked to the second transcription control element; incubating the transfected cells in a selective growth medium; isolating single cell clones from the resulting polyclonal, edited, and selected polyclonal cell population; amplifying at least one of the isolated single cell clones; Assessing Genome Editing in at least one amplified single cell clone; and obtaining a modified lepidopteran cell comprising a newly-introduced genome editing function resulting in a modified cellular phenotype.

According to certain embodiments, kits are provided. In certain embodiments, kits comprise a DNA vector of the current teachings comprising a lepidopteran insect U6 promoter and cells derived from a lepidopteran insect.

### BRIEF DESCRIPTION OF THE FIGURES

These and other features and advantages of the current teachings will become better understood with regard to the following description, appended claims, and accompanying figures. The skilled artisan will understand that the figures, described below, are for illustration purposes only. The figures are not intended to limit the scope of the disclosed teachings in any way.

FIGS. 1A-1D. Dm and Bm U6 promoters do not support CRISPR-Cas9 editing in Sf9 cells. FIG. 1A schematically depicts a generic CRISPR-Cas9 vector encoding, left to right, Streptococcus pyogenes Cas9 sequence codon optimized for Spodoptera frugiperda (SpCas9) under the control of a baculovirus ie1 promoter, an sgRNA expression cassette comprising an insect species-specific U6 promoter and a targeting sequence cloning site comprising two SapI recognition sites, and sequence encoding a puromycin resistance marker codon optimized for S. frugiperda under the control of baculovirus hr5 enhancer and ie1 promoter elements. FIG. 1B schematically depicts the Sf-fdl gene structure and highlights specific Cas9 targeting sequences (shown in Table 1) and PCR primer sites. FIGS. 1C and 1D depict CEL-I nuclease assay results obtained using genomic DNA from Sf9 cells edited with CRISPR-Cas9 vectors encoding various Sf-fdl targeting sequences (FIG. 1C: SfFDLt1 and SfFDLt2; FIG. 1D: SfFDLt3; shown in Table 1) under the control of either the DmU6:96Ab or the BmU6-2 promoter.

FIGS. 2A-D. CRISPR-Cas9 editing of fdl in S2R+ and BmN cells. FIG. 2A schematically depicts the Dm fdl gene. FIG. 2B depicts the CEL-I nuclease assay results obtained using a CRISPR-Cas9 vector of the current teachings comprising DmU6. These results demonstrate effective CRISPR-Cas9 editing of the Dm fdl gene with the vector comprising the DmU6 promoter. FIG. 2C schematically depicts the Bm fdl gene. FIG. 2D depicts the CEL-I nuclease assay results obtained using a CRISPR-Cas9 vector of the current teachings comprising BmU6. These results demonstrate effective CRISPR-Cas9 editing of the Bm fdl gene with the vector comprising the BmU6 promoter.

FIGS. 3A-C. Identification of putative SfU6 promoters and successful CRISPR-Cas9 editing of Sf-fdl. FIG. 3A depicts a multiple sequence alignment of BmU6-2 promoter (SEQ ID NO:29) and SfU6 promoter candidates SfU6-1, SfU6-2, SfU6-3, SfU6-4, SfU6-5, and SfU6-6 (SEQ ID NOs: 30-35, respectively). FIGS. 3B and 3C depict CEL-I nuclease assay results obtained using genomic DNA from Sf9 cells edited with CRISPR-Cas9 vectors encoding Sf-fdl targeting sequences (shown in Table 1) under the control of the BmU6-2 or SfU6-3 promoters.

FIGS. 4A-C. Sequences of Sf-fdl amplification products from Sf9 cells or Sf9 cells transfected with SfU6 CRISPR-Cas vectors encoding SfFDLt1 (FIG. 4A), SfFDLt2 (FIG. 4B), or SfFDLt3 (FIG. 4C) and selected for puromycin resistance

FIGS. 5A-C. Identification of putative TnU6 promoters and successful CRISPR-Cas9 editing of Tn-fdl. FIG. 5A depicts a multiple sequence alignment of SfU6 (SEQ ID NO: 36) and TnU6 promoter candidates TnU6-1, TnU6-2, TnU6-3, TnU6-4, TnU6-5, TnU6-6, TnU6-7, TnU6-8, TnU6-9, TnU6-10, and TnU6-11 (SEQ ID NOs: 37-44 and 53-55, respectively). FIG. 5B schematically depicts the Tn-fdl gene structure and highlights specific Cas9 targeting sequences and PCR primer sites. FIG. 5C depicts CEL-I nuclease assay results obtained using genomic DNA from HIGH FIVE™ cells edited with CRISPR-Cas9 vectors encoding a Tn-fdl targeting sequence (shown in Table 1) under the control of the DmU6:96Ab, BmU6-2, SfU6, or TnU6 promoters.

FIGS. 6A-D. CRISPR-Cas9 editing efficiencies by various insect U6 promoters in various insect cell lines. Various insect cell lines were transfected with DmU6:96Ab, SfU6, TnU6-4, and BmU6-2 CRISPR-Cas9 vectors encoding an EGFP-specific sgRNA, selected for puromycin resistance, and EGFP was measured by flow cytometry (the bars show mean fluorescence±s.d., n=3 per group). FIG. 6A graphically depicts results obtained with transfected S2R+-EGFP cells. FIG. 6B graphically depicts results obtained with transfected Sf9-EGFP cells. FIG. 6C graphically depicts results obtained with transfected HIGH FIVE™-EGFP cells. FIG. 6D graphically depicts results obtained with transfected BmN-EGFP cells.

FIG. 7 depicts the results of CEL-I nuclease assays demonstrating Sf-fdl indels in SfFDLt1 clones.

FIGS. 8A-8D graphically depicts the broader distribution of all indels, determined by TIDE analysis, in four clones determined to have no wild-type sequences or potentially functional in-frame deletions. The four clones are clone SfFDLt #4 (FIG. 8A), clone SfFDLt #14 (FIG. 8B), clone SfFDLt #32 (FIG. 8C), and clone SfFDLt #49 (FIG. 8D).

FIG. 9. Impact of Sf-fdl editing on N-glycan processing. N-glycans were isolated from hEPO produced by Sf9 (top panel), SfFDLt1 polyclonal (middle panel), or SfFDLt1 monoclonal cl#32 cells (lower panel), derivatized, and profiled by MALDI-TOF-MS, as depicted in FIG. 9. All molecular ions were detected as [M+Na]+, assigned and annotated using the standard cartoon symbolic representations.

FIG. 10. CRISPR-Cas9-mediated Sf-fdl gene editing for host engineering in the BICS. The bar graph shows the relative proportions of different N-glycan structures released from hEPO produced by Sf9, SfFDLt1 polyclonal population, and SfFDLt1 clone #32, as described. These data are derived from the MALDI-TOF-MS profiles depicted in FIG. 9 and represent the relative percentages of each N-glycan shown along the bottom of the Figure as a percentage of total.

FIG. 11. CRISPR-Cas9 editing efficiencies provided by various TnU6 promoters. High Five®-EGFP cells were transfected with TnU6-2, TnU6-3, TnU6-4, TnU6-5, TnU6-9, TnU6-10, TnU6-11, or SfU6-3 CRISPR-Cas9 vectors encoding an EGFP-specific sgRNA, selected for puromycin resistance, and EGFP was measured by flow cytometry (the bars show mean fluorescence±s.d., n=3 per group).

### DETAILED DESCRIPTION OF CERTAIN EXEMPLARY EMBODIMENTS

It is to be understood that both the foregoing general description and the following detailed descriptions are illustrative and exemplary only and are not intended to limit the scope of the disclosed teachings. The section headings used herein are for organizational purposes only and are not to be construed as limiting the subject matter of the disclosed teachings.

In the Summary above, the Detailed Description, the accompanying figures, and the claims below, reference is made to particular features (including method steps) of the current teachings. It is to be understood that the disclosure in this specification includes possible combinations of such particular features. For example, where a particular feature is disclosed in the context of a particular embodiment of the current teachings, or a particular claim, that feature can also be used, to the extent possible, in combination with and/or in the context of other particular embodiments, and in the current teachings in general.

Where reference is made to a method comprising two or more combined steps, the defined steps can be performed in any order or simultaneously (except where the context excludes that possibility), and the method include one or more other steps which are carried out before any of the defined steps, between two of the defined steps, or after all of the defined steps (except where the context excludes that possibility).

### Definitions

As used in this description and in the appended claims, the term “Assessing Genome Editing” is used in a broad sense and is intended to encompass a wide variety of techniques that are, or could be, used to evaluate whether or not genome editing occurred and produced the desired cellular phenotype in a population of cells. For example but not limited to, a cell that has been transformed with a DNA vector of the current teachings. The person of ordinary skill in the art will readily be able to evaluate whether genome editing is occurring or not using one or more technique known in the art. Exemplary techniques suitable for Assessing Genome Editing include but are not limited to CEL-I nuclease assay, DNA sequencing with TIDE analysis, PCR followed by cloning and sequencing individual clones, and phenotypic assays, such as polyacrylamide gel electrophoresis, western blotting, ELISA, gel shift assays, glycan profiling, phosphate profiling, lipid profiling and mass spectrometry, including without limitation MALDI-TOF-MS profiling of glycan structures.

As used in this description and in the appended claims, the term “comprising”, which is synonymous with “including”, and cognates of each (such as comprise, comprises, include, and includes), is inclusive or open-ended and does not exclude additional unrecited components, elements, or method steps; that is, other components, steps, etc., are optionally present. For example but not limited to, an article “comprising” components A, B, and C may consist of (that is, contain only) components A, B, and C; or the article may contain not only components A, B, and C, but also one or more additional components.

As used in this description and in the appended claims, the term “or combinations thereof” refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, ACB, CBA, BCA, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

As used in this description and in the appended claims, a coding sequence and a transcription control element are said to be “operably linked” when they are covalently linked in such a way as to place the expression or transcription and/or translation of the coding sequence under the influence or control of the transcription control element. A “transcription control element” may be any nucleic acid element, including but not limited to promoters, enhancers, transcription factor binding sites, polyadenylation signals, termination signals, and other elements that direct the expression of a nucleic acid sequence or coding sequence that is operably linked thereto.

As used in this description and in the appended claims, a “selectable marker” is a coding sequence that, when expressed, may confer in the cell in which it has been transfected, the ability to survive or provide resistance to antibiotics or toxins, complement auxotrophic deficiencies, or supply critical nutrients not present in the culture media. Selectable markers often comprise antibiotic resistance genes. Cells that have been transfected with a selectable marker conferring antibiotic resistance are grown in a selective medium that contains the corresponding antibiotic. The antibiotic kills those cells that do not have the selectable marker. Those cells that can grow have successfully taken up and expressed the selectable marker, and are thus resistant to the antibiotic in the medium. For example, a cell transfected with certain disclosed DNA vectors comprise a puromycin resistance marker (puromycin acetyl transferase, pac) under the control of a third transcription control element, comprising, for example, a baculovirus hr5 enhancer and ie1 promoter elements. When such transfected cells express sufficient quantities of puromycin acetyl transferase, they can survive in selective media comprising the antibiotic puromycin; while untransfected cells will die due to the presence of puromycin. Exemplary selectable markers that may be suitable for use in the disclosed DNA vectors include coding sequences that confer resistance to puromycin, blasticidin S, G418, hygromycin, zeocin, and nouroseothricin.

As used in this description and in the appended claims, the term “targeting sequence” refers to a geneecific sequence approximately 20 base pairs long that is selected to be complementary to the DNA sequence to be edited. Exemplary targeting sequences include, but are not limited to, SEQ ID NO:1, which targets the FDL gene of Drosophila melanogaster; SEQ ID NOs: 2-4, which target the FDL gene of Spodoptera frugiperda; SEQ ID NOs: 6-8, which target the FDL gene of Bombyx mori; and SEQ ID NO:9, which targets the EGFP gene.

The clustered, regularly interspaced, short palindromic repeat (CRISPR)-Cas9 system is a relatively new and exceptionally powerful tool for site-specific genome editing. CRISPR-Cas9 vectors have been constructed for and used in many different biological systems, including insect cell systems. In fact, it has been shown that endogenous U6 promoters can be used to drive single guide RNA (sgRNA) expression for CRISPR-Cas9 genome editing in S2R+, a cell line derived from the dipteran insect, Drosophila melanogaster (Dm) and BmN, a cell line derived from the lepidopteran insect, Bombyx mori (Bm). These findings prompted us to attempt to adopt the CRISPR-Cas9 system for site-specific genome editing in the BICS. The broader purpose of this effort was to provide enabling technology for precise genetic modifications that will further enhance and expand the utility of this important recombinant protein production platform. Surprisingly, we found previously described insect U6 promoters failed to support CRISPR-Cas9 editing in lepidopteran insect cell systems.

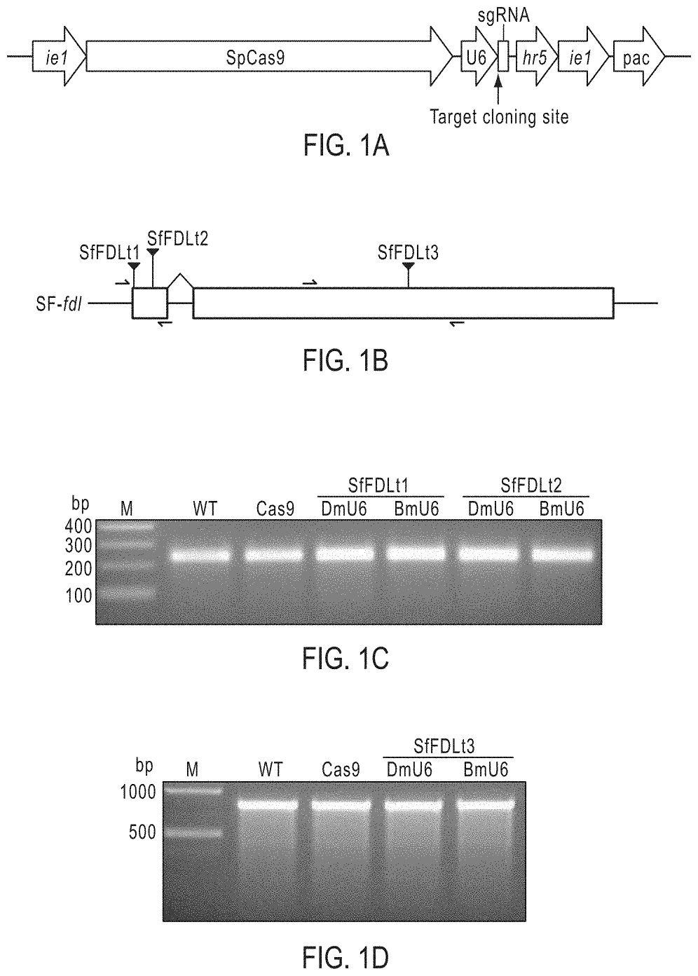

FIG. 1A provides a schematic overview of certain exemplary CRISPR-Cas9 of the current teachings. Genetically engineered “generic” CRISPR-Cas9 vectors were designed to include an Sf codon-optimized Streptococcus pyogenes (Sp) Cas9 coding sequence under the control of a baculovirus ie1 promoter, which provides constitutive transcription in a wide variety of organisms, followed by a U6 promoter, including but not limited to, the DmU6:96Ab or the BmU6-2 promoter, for sgRNA expression, and a targeting sequence cloning site. These vectors also included a puromycin resistance marker (puromycin acetyl transferase, pac) under the control of baculovirus hr5 enhancer and ie1 promoter elements. These operationally linked elements are depicted in FIG. 1A, as ie1-SpCas9-U6-targeting sequence cloning site-sgRNA-hr5-ie1-pac, left to right. According to certain DNA vector embodiments, the SpCas 9 coding sequence was not Sf codon optimized, the selectable marker was not Sf codon optimized, or both the SpCas9 coding sequence and the selectable marker were not Sf codon optimized. In certain embodiments, the SpCas9 coding sequence is codon optimized for Spodoptera frugiperda, the selectable marker is codon optimized for Spodoptera frugiperda, or both the SpCas9 coding sequence and the selectable marker are codon optimized for Spodoptera frugiperda.

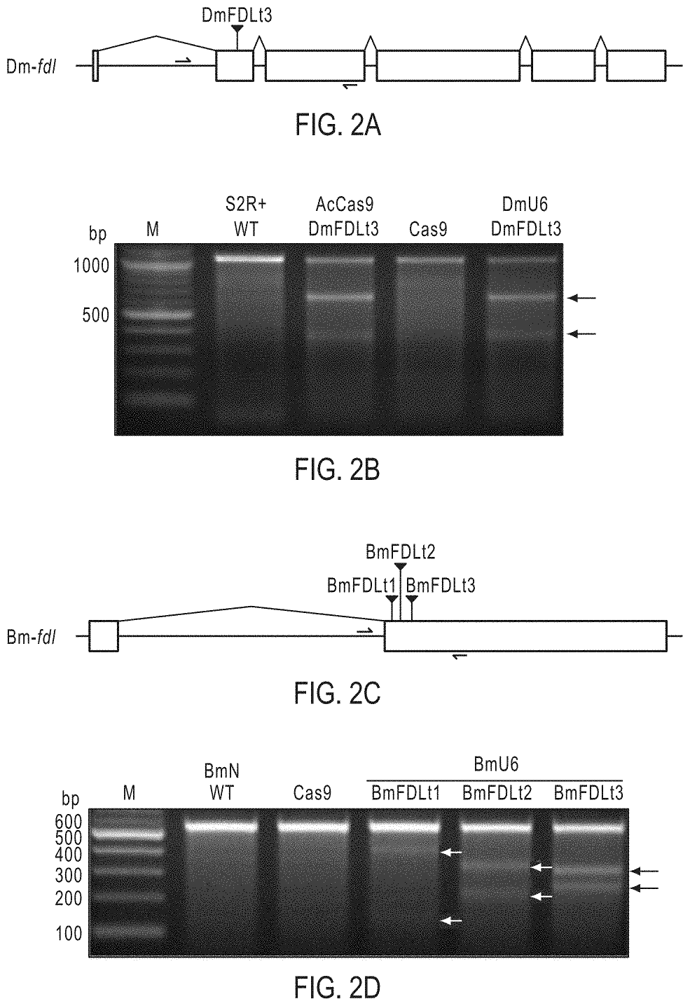

In certain exemplary CRISPR-Cas9 vector embodiments, targeting sequences for the Dm or Bm fdl genes (FIGS. 2A and 2C, respectively) were inserted into a generic vector. The editing capacity of such constructs were evaluated by transfecting the construct into insect cell lines and determining whether the fdl genes in the transfected cells were efficiently edited using CEL-I nuclease assays on puromycin resistant derivatives (for example, as shown in FIGS. 2B and 2D).

According to the current teachings, various insect U6 promoters were used to construct novel CRISPR-Cas9 vectors, similar to the construct depicted in FIG. 1A. The utility of the disclosed novel CRISPR-Cas9 vectors for site-specific genome editing was demonstrated in two insect cell lines commonly used as hosts in the BICS, Sf and Tn. We discovered that, unlike constructs containing previously described Dm and Bm U6 promoters, our novel CRISPR-Cas9 vector constructs were able to edit an endogenous insect cell gene and alter protein glycosylation in the BICS. The novel tools disclosed herein will enable new efforts to enhance the capabilities and expand the utility of this important protein production platform.

According to certain DNA vector embodiments, a first expression cassette comprises a CRISPR-associated endonuclease coding sequence operably linked to a first transcription control element. Typically, the first transcription control element is capable of driving constitutive expression of the CRISPR-associated endonuclease coding sequence at levels that support efficient CRISPR-Cas-mediated genome editing in insect cells. In certain embodiments, the first transcription control element comprises a baculovirus immediate early promoter, a baculovirus early promoter, a baculovirus enhancer, a polyadenylation signal, or combinations thereof. In certain embodiments, the first transcription control element comprises a baculovirus ie1 promoter, a baculovirus ie2 promoter, a baculovirus ie0 promoter, a baculovirus etl promoter, a baculovirus gp64 promoter, a baculovirus hr1 enhancer, a baculovirus hr2 enhancer, a baculovirus hr3 enhancer, a baculovirus hr4 enhancer, a baculovirus hr5 enhancer, a p10 polyadenylation signal, or combinations thereof. In certain embodiments, the CRISPR-associated endonuclease coding sequence comprises the Streptococcus pyogenes Cas9 (SpCas9) sequence. In certain embodiments, the SpCas9 coding sequence is codon optimized for Spodoptera frugiperda.

According to certain embodiments, a second expression cassette comprises a targeting sequence cloning site and a sgRNA coding sequence operably linked to the second transcription control element, wherein the targeting sequence cloning site is inserted between the second transcription control element and the sgRNA coding sequence. In certain embodiments, the second transcription control element is capable of driving targeting sequence (when a targeting sequence is inserted into the targeting sequence cloning site) and sgRNA expression at levels that support efficient CRISPR-Cas-mediated genome editing in insect cells. In certain embodiments, the second transcription control element comprises a U6 promoter from a lepidopteran insect. In certain embodiments, the second transcription control element comprises a U6 promoter derived from Spodoptera frugiperda or Trichoplusia ni.

In certain embodiments, the targeting sequence cloning site enables insertion of a targeting sequence needed to direct efficient site-specific editing in insect cells. In certain embodiments, the targeting sequence cloning site of the sgRNA expression cassette comprises two adjacent type IIS restriction endonuclease sites. In certain embodiments, the targeting sequence cloning site comprises at least one SapI recognition site. In certain embodiments, the targeting sequence cloning site comprises two adjacent SapI recognition sites. In certain embodiments, the DNA vector further comprises a targeting sequence inserted into the targeting sequence insertion site and operably linked to a second transcriptional control element. In certain embodiments, the inserted targeting sequence comprises SEQ ID NO:1, SEQ ID NO:2, SEQ ID NO:3, SEQ ID NO:4, SEQ ID NO:5, SEQ ID NO:6, SEQ ID NO:7, SEQ ID NO:8, or SEQ ID NO:9.

Those in the art will appreciate that the selected sgRNA coding sequence mediates efficient site-specific editing in insect cells. In certain embodiments, the sgRNA coding sequence comprises:

(SEQ ID NO: 45)
GTTTTAGAGCTAGAAATAGCAAGTTAAAATAAGGCTAGTCCGTTATCAAC
TTGAAAAAGTGGCACCGAGTCGGTGCTTTTTT.

In certain embodiments, the third expression cassette comprises a selectable marker operably linked to the third transcription control element. In certain embodiments, the selectable marker comprises a puromycin, blasticidin S, G418, hygromycin, zeocin, or nourseothricin resistance marker. In certain embodiments, the selectable marker comprises the sequence encoding puromycin acetyl transferase (pac). In certain embodiments, the sequence encoding puromycin acetyl transferase is under the control of baculovirus hr5 enhancer and ie1 promoter elements. In certain embodiments, the third transcription control element comprises a baculovirus promoter, a Respiratory Syncytial Virus (RSV) promoter, a copia promoter, a gypsy promoter, a piggyBac promoter, a cytomegalovirus immediate early promoter, a baculovirus enhancer, or combinations thereof. In certain embodiments, the third transcription control element comprises a baculovirus ie1 promoter and a baculovirus hr5 enhancer. It is understood by those in the art that the third transcription control element should be capable of driving constitutive expression of the selectable marker sequence at levels that produce resistance in a specific selection protocol. In certain embodiments, the selectable marker is Sf codon optimized.

In certain vector embodiments, the Cas9 expression cassette comprises the SpCas9 coding sequence codon optimized for S. frugiperda and the first transcription control element comprises a baculovirus ie1 promoter and a p10 polyadenylation signal; the sgRNA expression cassette comprises a target sequence cloning site, the sgRNA coding sequence comprises SEQ ID NO: 45, and the second transcription control element comprises a lepidopteran U6 promoter; and the selectable marker expression cassette comprises a sequence encoding puromycin acetyl transferase which is codon optimized for S. frugiperda, and the third transcription control element comprises a baculovirus ie1 promoter, a baculovirus hr5 enhancer, and a baculovirus p10 polyadenylation signal (see, e.g., FIG. 1A and SEQ ID NO: 46).

According to certain embodiments, insect cells transformed with vectors of the current teachings are provided. In certain embodiments, the cell is derived from a lepidopteran insect. In certain embodiments, the insect cell is derived from Spodoptera frugiperda, Trichoplusia ni or Bombyx mori. In certain embodiments, the insect cell is derived from Sf-RVN cells, Sf9 cells, Sf21 cells, EXPRESSF+® cells, SUPER 9® cells, Tn-NVN cells, Tn368 cells, HIGH FIVE® cells, TNI PRO® cells, Ea4 cells, BTI-Tnao38 cells, or BmN cells.

According to certain embodiments, lepidopteran insect cells are provided, wherein the FDL function in the cells is reduced enough to reduce the cells ability to synthesize insect-type, paucimannosidic N-glycans (M3Gn2+/−Fuc) to less than 10% of total, as determined by MALDI-TOF-MS profiling of glycan structures.

According to certain embodiments, methods for obtaining a modified lepidopteran cell comprising a newly-introduced genome editing function resulting in a modified cellular phenotype are provided. In certain embodiments, the methods comprise:

transfecting a lepidopteran insect cell with a DNA vector comprising: a Streptococcus pyogenes Cas9 (SpCas9) coding sequence operably linked to a first transcriptional control element; a single guide RNA (sgRNA) expression cassette comprising a targeting sequence cloning site, a targeting sequence, and a sgRNA coding sequence operably linked to a second transcriptional control element; and a selectable marker operably linked to a third transcriptional control element;

incubating the transfected cells in a selective growth medium;

isolating single cell clones from the resulting polyclonal edited, selected polyclonal cell population;

amplifying at least one of the isolated single cell clones;

Assessing Genome Editing in at least one amplified single cell clone; and

obtaining a modified lepidopteran cell comprising a newly-introduced genome editing function resulting in a modified cellular phenotype.

In certain embodiments, methods for obtaining a modified lepidopteran cell comprising a newly-introduced genome editing function resulting in a modified cellular phenotype comprise:

transfecting a lepidopteran insect cell with a DNA vector, wherein the sgRNA expression cassette of the vector further comprises a targeting sequence;

incubating the transfected cells in a selective growth medium;

isolating single cell clones from the resulting polyclonal edited, selected polyclonal cell population;

amplifying at least one of the isolated single cell clones;

Assessing Genome Editing in at least one amplified single cell clone; and

obtaining a modified lepidopteran cell comprising a newly-introduced genome editing function resulting in a modified cellular phenotype.

In certain method embodiments, the DNA vector comprises the DNA vector of claim 4, further comprising targeting sequence SEQ ID NO: 2 inserted in the targeting sequence cloning site and operably linked to the second transcription control element. In certain embodiments, the DNA vector comprises the vector of claim 19, wherein the DNA vector further comprises SEQ ID NO: 2 inserted in the targeting sequence cloning site and operably linked to the second transcription control element.

According to certain embodiments, insect cells transformed with a disclosed DNA vector are provided. In certain embodiments, the insect cell is transformed with the DNA vector of claim 1, wherein the insect cell is derived from Spodoptera frugiperda, Trichoplusia ni or Bombyx mori. In certain embodiments, the insect cell is transformed with the DNA vector of claim any of the DNA vectors of claim 2-18, wherein the insect cell is derived from Spodoptera frugiperda, Trichoplusia ni or Bombyx mori. In certain embodiments, such insect cells are derived from Sf-RVN cells, Sf9 cells, Sf21 cells, EXPRESSF+® cells, Tn-NVN cells, Tn368 cells, HIGH FIVE® cells, TNI PRO® cells, Ea4 cells, BTI-Tnao38 cells, or BmN cells.

According to certain embodiments, a lepidopteran insect cell produced by certain disclosed methods comprising a newly-introduced genome editing function comprises reducing FDL function enough to reduce the cells ability to synthesize insect-type, paucimannosidic N-glycans (M3Gn2+/−Fuc) to less than 10% of total, as determined by MALDI-TOF-MS profiling of glycan structures.

According to certain embodiments, a lepidopteran insect cell wherein FDL function is reduced enough to reduce the cells ability to synthesize insect-type, paucimannosidic N-glycans (M3Gn2+/−Fuc) to less than 10% of total, as determined by MALDI-TOF-MS profiling of glycan structures is provided.

### Certain Exemplary Techniques

Cells. S2R+ cells were maintained at 28° C. as adherent cultures in Schneider's Drosophila medium (Life Technologies) containing 10% (v/v) fetal bovine serum (Atlanta Biologics). Sf9, HIGH FIVE™, and BmN cells were maintained at 28° C. as adherent cultures in TNM-FH medium containing 10% (v/v) fetal bovine serum. Sf9 cells were transfected using a modified calcium phosphate method (8) and S2R+, HIGH FIVE®, and BmN cells were transfected with polyethyleneimine, as described previously (25). S2R-EGFP, Sf9-EGFP, Tn-EGFP, and BmN-EGFP cells are transgenic derivatives of S2R+, Sf9, HIGH FIVE®, and BmN cells, respectively, produced by transfecting each parental cell line with pIE1-EGFP-Bla and selecting for blasticidin resistance. Blasticidin-resistant cells expressing EGFP in the top quartile were isolated using a MOFLO™ Legacy Cell Sorter (Beckman Coulter) and enriched cell subpopulations were maintained under the same growth conditions as the parental cell lines.

Plasmid Constructions. All CRISPR-Cas9 constructs were generically designed to include three distinct cassettes for expression of Cas9, an sgRNA, and a puromycin resistance marker (for example, as shown in FIG. 1A). The Cas9 expression cassette consists of a S. pyogenes Cas9 sequence codon optimized for S. frugiperda and assembled with the AcMNPV ie1 promoter and p10 polyadenylation signal using the Golden Gate method. The sgRNA expression cassettes consist of DmU6:96Ab, BmU6-2, SfU6-3, TnU6-2, TnU6-3, TnU6-4, TnU6-5, TnU6-6, TnU6-7, TnU6-8, TnU6-9, TnU6-10, or TnU6-11 promoters assembled with various downstream sgRNA sequences. The targeting sequences incorporated into various sgRNAs are provided in Table 1. A targeting sequence cloning site comprising two SapI recognition sites was inserted between the U6 promoter and sgRNA in each CRISPR-Cas9 plasmid. Finally, the puromycin resistance marker was codon optimized for S. frugiperda and assembled with the AcMNPV ie1 promoter and p10 polyadenylation signal. The nucleotide sequences for a generic DNA vector of the current teachings (based on pIE1-Cas9-DmU6-sgRNA-Puro) and the specific U6 promoters in each of the other DNA vectors used in the current teachings are provided in SEQ ID NOs: 46 (plasmid) and 47-55 (specific U6 promoters). The generic plasmid (SEQ ID NO:46) comprises the DmU6 promoter. According to the current teachings, to obtain a modified lepidopteran cell comprising a newly-introduced genome editing function resulting in a modified cellular phenotype, the DmU6 promoter must be replaced by an appropriate lepidopteran U6 promoter (SEQ ID NOS: 47-55) and an appropriate targeting sequence.

TABLE 1
sgRNA targeting sequences used in this study.

| Name of | | |
|---|---|---|
| target | Target | Sequence |
| site | gene | (5′ to 3′) |
| DmFDLt3 | Dm-fdl | gcgccatattcatcctga |
| | | (SEQ ID NO: 1) |
| SfFDLt1 | Sf-fdl | ggcagtgcgatgaagtgg |
| | | (SEQ ID NO: 2) |
| SfFDLt2 | Sf-fdl | gccgcggcgctgctgtac |
| | | (SEQ ID NO: 3) |
| SfFDLt3 | Sf-fdl | gaagtgtcggaacgttgc |
| | | (SEQ ID NO: 4) |
| TnFDLt | Tn-fdl | gaagtgtccgagcgctgc |
| | | (SEQ ID NO: 5) |
| BmFDLt1 | Bm-fdl | gcgagaggtatcaagcat |
| | | (SEQ ID NO: 6) |
| BmFDLt2 | Bm-fdl | gctctggccacagccgac |
| | | (SEQ ID NO: 7) |
| BmFDLt3 | Bm-fdl | ggcctgtcagcctcgcat |
| | | (SEQ ID NO: 8) |
| EGFPt | EGFP | gggcgaggagctgttcac |
| | | (SEQ ID NO: 9) |

The nucleotide sequence of the generic plasmid (pIE1-Cas9-DmU6-sgRNA-Puro), is shown below; the DmU6 promoter sequence is underlined:

(SEQ ID NO: 46)
tcgatgtctttgtgatgcgcgcgacatttttgtaggttattgataaaatgaacggatacgttgcccgacattatcattaaatccttggcgtagaattt
gtcgggtccattgtccgtgtgcgctagcatgcccgtaacggacctcgtacttttggcttcaaaggttttgcgcacagacaaaatgtgccacact
tgcagctctgcatgtgtgcgcgttaccacaaatcccaacggcgcagtgtacttgttgtatgcaaataaatctcgataaaggcgcggcgcgcg
aatgcagctgatcacgtacgctcctcgtgttccgttcaaggacggtgttatcgacctcagattaatgtttatcggccgactgttttcgtatccgct
caccaaacgcgtttttgcattaacattgtatgtcggcggatgttctatatctaatttgaataaataaacgataaccgcgttggttttagagggcata
ataaaagaaatattgttatcgtgttcgccattagggcagtataaattgacgttcatgttggatattgtttcagttgcaagttgacactggcggcga
caagatcgtgaacaaccaagtgacaacatggactacaaggaccacgacggcgattacaaggatcacgacatcgactacaaggacgatga
cgacaagatggcccccaagaagaagcgcaaagtcggtatccacggtgtccccgctgctgacaagaagtactccatcggcctggacatcg
gcaccaactccgtgggctgggctgtgatcaccgacgagtacaaggtgccctccaagaagttcaaggtcctgggcaacaccgaccgtcact
ccatcaagaagaacctgatcggcgctctgctgttcgactccggcgagactgctgaggctacccgtctgaagcgtaccgctcgtcgtcgttac
acccgtcgcaagaaccgtatctgctacctgcaagagatcttctccaacgagatggctaaggtggacgacagcttcttccaccgtctggaaga
gtccttcctggtggaagaggacaagaagcacgagcgtcaccccatcttcggcaacatcgtggacgaggtggcctaccacgagaagtacc
ccaccatctaccacctccgcaagaagctggtcgactccaccgacaaggctgacctgcgtctgatctacctggctctggctcacatgatcaag
ttccgtggtcacttcctgatcgagggcgacctgaaccccgacaactccgacgtggacaagctgttcatccagctggtgcagacctacaacc
agctgttcgaggaaaaccccatcaacgcttccggtgtcgacgctaaggctatcctgtccgctcgtctgtccaagtcccgtcgtctggaaaact
tgatcgctcagctgcccggcgagaagaagaacggcctgttcggcaacctgatcgctctgtccctgggcctgacccccaacttcaagtccaa
cttcgacctggctgaggacgctaagctccagctgtccaaggacacctacgacgatgacctggacaacctgctggctcagatcggcgacca
gtacgctgacctgttcctggctgctaagaacctgtccgacgctatcctgctgtccgacatcctgcgtgtgaacaccgagatcaccaaggctc
ctctgtccgcttctatgatcaagcgttacgacgagcaccaccaggacctgaccctgctgaaggctctcgtgcgtcagcagctgcctgagaa
gtacaaggaaatcttcttcgaccagtccaagaacggctacgctggttacatcgacggtggtgcttcccaagaggaattctacaagttcatcaa
gcccatcctcgagaagatggacggcaccgaggaactgctggtcaagctgaaccgcgaggacctgctgcgcaagcagcgcaccttcgac
aacggttccatcccccaccagatccacctgggcgagttgcacgctatcttgcgtcgtcaagaggacttctacccattcctgaaggacaaccg
cgagaagatcgaaaagatcctgaccttccgtatcccctactacgtgggtcccctggctcgtggcaactcccgtttcgcttggatgacccgca
agtccgaggaaaccatcaccccctggaacttcgaagaggtggtggacaagggcgcttccgctcagtccttcatcgagcgtatgactaactt
cgacaagaacctgcccaacgagaaggtgctgcccaagcactccctgctgtacgagtacttcaccgtgtacaacgagctgaccaaagttaa
atacgtgaccgagggaatgcgcaagcccgctttcctgtccggcgagcaaaagaaggctatcgtcgacctgctgttcaagaccaaccgcaa
agtgaccgtgaagcagctgaaggaagattacttcaagaagatcgagtgcttcgacagcgtcgagatctccggcgtcgaggaccgtttcaac
gcctccctgggcacttaccacgacctgctcaagatcatcaaggacaaggatttcttggacaacgaagagaacgaggacatcttggaggac
atcgtgctgaccctgaccctcttcgaggacagagagatgatcgaggaacgcctcaagacctacgctcacttgttcgacgacaaagtgatga
agcaactcaagcgtcgccgctacaccggctggggtcgtctgtctcgcaagctgatcaacggtatccgtgacaagcagtccggcaagactat
cctggacttcctgaagtccgacggtttcgctaaccgtaacttcatgcagctgatccacgacgactccctgactttcaaggaggacatccaaaa
ggctcaggtgtccggccagggcgactctctgcacgagcacatcgctaacctggctggttcccccgctatcaagaagggtatcctgcagac
cgtcaaggtggtcgacgaactggtcaaagtcatgggtcgtcacaagcccgagaacatcgtcatcgagatggcccgcgagaaccagacca
cccagaagggtcaaaagaactcccgcgagcgcatgaagcgtatcgaagaaggcatcaaggaactgggttcccagatcctcaaggaaca
ccccgtcgagaacacccagctgcagaacgagaagctgtacctgtactacctccagaacggtcgcgatatgtacgtggaccaagagctgga
catcaaccgtctgtccgactacgatgtcgaccacatcgtgccccagtctttcttgaaggacgactcgatcgacaacaaggtgctgactcgttc
cgataagaaccgtggaaagtccgacaacgtcccctccgaagaggtcgtgaagaagatgaagaactactggcgtcagctgctcaacgcca
agctcatcacccagaggaagttcgacaacttgaccaaggctgagcgtggtggcctgtccgaactggacaaggccggtttcatcaagaggc
agctggtggaaacccgtcagatcactaagcacgtggcccagatcttggactcccgtatgaacactaagtacgacgagaacgacaagttgat
ccgcgaagtgaaagtgatcaccctcaagtctaagctggtgtccgacttccgcaaggacttccagttctacaaagtgcgcgagatcaacaact
accaccacgcccacgacgcttacctgaacgctgtcgtgggcaccgccctcatcaagaagtaccctaagctcgagtccgagttcgtgtacgg
cgactacaaggtgtacgacgtgcgcaagatgatcgctaagtccgagcaagaaatcggcaaggctaccgccaagtacttcttctactccaac
atcatgaacttcttcaagactgagatcaccctggccaacggcgagatccgcaagcgtcctctgatcgagactaacggcgaaactggcgag
atcgtgtgggacaagggtcgtgacttcgctaccgtcagaaaggtgctgtccatgccccaagtgaacatcgttaagaagaccgaggtccaga
ccggtggtttctccaaggaatccatcctgcctaagaggaactccgataagctgatcgctaggaagaaggactgggaccctaagaagtacg
gcggtttcgactcccccaccgtggcttactctgtgctggtggtcgctaaggtcgagaagggaaagtctaagaagctcaagtccgtcaagga
attgctgggcatcaccatcatggaacgctccagcttcgagaagaaccctatcgacttcctcgaggctaagggctacaaggaagtcaagaag
gacctcatcatcaagctccccaagtacagcctgttcgagctggaaaacggtcgcaagcgtatgctggcttccgctggcgaactgcagaagg
gcaacgaactggctctgccctctaaatacgtcaacttcctgtacctggcttcccactacgaaaagctgaagggctcccccgaggataacgaa
caaaagcaactgttcgtcgagcagcacaagcactacctggacgagatcatcgagcagatctccgagttctccaagcgtgtgatcctggctg
acgctaacctcgataaggtgctctccgcttacaacaagcaccgcgacaagcctatccgcgagcaggctgagaacatcatccacctgttcac
cctgactaacctgggtgctcccgctgctttcaagtacttcgacaccaccatcgaccgcaagcgctacacctccaccaaggaagtgctcgac
gctaccctgatccaccagtccatcaccggcctgtacgagactcgtatcgacctgtcccagctcggtggcgacaagcgtccagctgctacca
agaaggctggccaggctaagaagaagtaatgtaaacgccacaattgtgtttgttgcaaataaacccatgattatttgattaaaattgttgttttctt
tgttcatagacaatagtgtgttttgcctaaacggtttgggagatctaagcttcatatggtgcactctcagtacaatctgctctgatgccgcatagtt
aagccagccccgacacccgccaacacccgctgacgcgccctgacgggcttgtctgctcccggcatccgcttacagacaagctgtgaccg
tctccgggagctgcatgtgtcagaggttttcaccgtcatcaccgaaacgcgcgagacgaaagggcctcgtgatacgcctatttttataggtta
atgtcatgataataatggtttcttagacgtcaggtggcacttttcggggaaatgtgcgcggaacccctatttgtttatttttctaaatacattcaaat
atgtatccgctcatgagacaataaccctgataaatgcttcaataatattgaaaaaggaagagtatgagtattcaacatttccgtgtcgcccttatt
cccttttttgcggcattttgccttcctgtttttgctcacccagaaacgctggtgaaagtaaaagatgctgaagatcagttgggtgcacgagtggg
ttacatcgaactggatctcaacagcggtaagatccttgagagttttcgccccgaagaacgttttccaatgatgagcacttttaaagttctgctatg
tggcgcggtattatcccgtattgacgccgggcaagagcaactcggtcgccgcatacactattctcagaatgacttggttgagtactcaccagt
cacagaaaagcatcttacggatggcatgacagtaagagaattatgcagtgctgccataaccatgagtgataacactgcggccaacttacttct
gacaacgatcggaggaccgaaggagctaaccgcttttttgcacaacatgggggatcatgtaactcgccttgatcgttgggaaccggagctg
aatgaagccataccaaacgacgagcgtgacaccacgatgcctgtagcaatggcaacaacgttgcgcaaactattaactggcgaactactta
ctctagcttcccggcaacaattaatagactggatggaggcggataaagttgcaggaccacttctgcgctcggcccttccggctggctggttta
ttgctgataaatctggagccggtgagcgtgggtctcgcggtatcattgcagcactggggccagatggtaagccctcccgtatcgtagttatct
acacgacggggagtcaggcaactatggatgaacgaaatagacagatcgctgagataggtgcctcactgattaagcattggtaactgtcaga
ccaagtttactcatatatactttagattgatttaaaacttcatttttaatttaaaaggatctaggtgaagatcctttttgataatctcatgaccaaaatcc
cttaacgtgagttttcgttccactgagcgtcagaccccgtagaaaagatcaaaggatcttcttgagatcctttttttctgcgcgtaatctgctgctt
gcaaacaaaaaaaccaccgctaccagcggtggtttgtttgccggatcaagagctaccaactctttttccgaaggtaactggcttcagcagag
cgcagataccaaatactgtccttctagtgtagccgtagttaggccaccacttcaagaactctgtagcaccgcctacatacctcgctctgctaat
cctgttaccagtggctgctgccagtggcgataagtcgtgtcttaccgggttggactcaagacgatagttaccggataaggcgcagcggtcg
ggctgaacggggggttcgtgcacacagcccagcttggagcgaacgacctacaccgaactgagatacctacagcgtgagctatgagaaag
cgccacgcttcccgaagggagaaaggcggacaggtatccggtaagcggcagggtcggaacaggagagcgcacgagggagcttccag
ggggaaacgcctggtatctttatagtcctgtcgggtttcgccacctctgacttgagcgtcgatttttgtgatgctcgtcaggggggcggagcct
atggaaaaacgccagcaacgcggcctttttacggttcctggccttttgctggccttttgctcacatgttctttcctgcgttatcccctgattctgtg
gataaccgtattaccgcctttgagtgagctgataccgctcgccgcagccgaacgaccgagcgcagcgagtcagtgagcgaggaagcgga
attggatcccgggccggctaattcgttcgacttgcagcctgaaatacggcacgagtaggaaaagccgagtcaaatgccgaatgcagagtct
cattacagcacaatcaactcaagaaaaactcgacacttttttaccatttgcacttaaatccttttttattcgttatgtatactttttttggtccctaacca
aaacaaaaccaaactctcttagtcgtgcctctatatttaaaactatcaatttattatagtcaataaatcgaactgtgttttcaacaaacgaacaata
ggacactttgattctaaaggaaattttgaaaatcttaagcagagggttcttaagaccatttgccaattcttataattctcaactgctctttcctgatgt
tgatcatttatataggtatgttttcctcaatacttcggaagagcgatatcaagcttggtacccaagctcttccgttttagagctagaaatagcaagt
taaaataaggctagtccgttatcaacttgaaaaagtggcaccgagtcggtgcttttttctgcagactagtgcggccgcaaatgtttgggccgc
gtaaaacacaatcaagtatgagtcataagctgatgtcatgttttgcacacggctcataaccgaactggctttacgagtagaattctacttgtaac
gcacgatcgagtggatgatggtcatttgtttttcaaatcgagatgatgtcatgttttgcacacgggctcataaactgctttacgagtagaattcta
cgtgtaacgcacgatcgattgatgagtcatttgttttgcaatatgatatcatacaatatgactcatttgtttttcaaaaccgaacttgatttacgggta
gaattctactcgtaaagcacaatcaaaaagatgatgtcatttgtttttcaaaactgaactctcggctttacgagtagaattctacgtgtaaaacac
aatcaagaaatgatgtcatttgttataaaaataaaagctgatgtcatgttttgcacatggctcataactaaactcgctttacgggtagaattctacg
tcgatgtctttgtgatgcgccgacatttttgtaggttattgataaaatgaacggatacagttgcccgacattatcattaaatccttggcgtagaattt
gtcgggtccattgtccgtgtgcgctagcatgcccgctaacggacctcgtacttttggcttcaaaggttttgcgcacagacaaaatgtgccaca
cttgcagctctgcatgtgtgcgcgttaccacaaatcccaacggcgcagtgtacttgttgtatgcaaataaatctcgataaaggcgcggcgcgc
gaatgcagctgatcacgtacgctcctcgtgttccgttcaaggacggtgttatcgacctcagattaatgtttatcggccgactgttttcgtatccgc
tcaccaaacgcgtttttgcattaacattgtatgtcggcggatgttctatatctaatttgaataaataaacgataaccgcgttggttttagagggcat
aataaaagaaatattgttatcgtgttcgccattagggcagtataaattgacgttcatgttggatattgtttcagttgcaagttgacactggcggcg
acaagatcgtgaacaaccaagtgacgcggatctagatctcgagcggccgcaccatgaccgagtacaagcccaccgtgcgtctggctacc
cgtgacgatgtgcctcgtgctgtgcgtaccctggctgctgctttcgctgactaccccgctacccgtcacaccgtggatcccgaccgtcacatc
gagcgtgtgaccgagctgcaagagctgttcctgacccgtgtgggcctggacatcggcaaagtgtgggtggccgacgacggtgctgctgtg
gctgtgtggaccacccctgagtccgtggaagctggtgctgtgttcgctgagatcggtccccgtatggctgagctgtccggttcccgtctggct
gctcagcagcagatggaaggcctgctggctccccaccgtcctaaggaacctgcctggttcctggctaccgtgggcgtgtcacctgaccac
cagggaaagggactgggttccgctgtggtgctgcctggtgtcgaggctgctgaacgtgctggtgtccccgctttcctggaaacctccgctcc
ccgtaacctgcccttctacgagcgtctgggtttcaccgtgaccgctgacgtggaagtgcccgagggtcctcgtacctggtgcatgactcgca
agcccggtgcttaagtttcgatgtaaacgccacaattgtgtttgttgcaaataaacccatgattatttgattaaaa.

Splinkerette PCR. Splinkerette PCR was performed using a known method. Briefly, Sf9 or HIGH FIVE™ genomic DNA was digested with BamHI, BglII, BstYI, HindIII, SalI, SpeI, or XbaI, and ligated with splinkerette adaptors complementary to the resulting overhangs. Primary and secondary PCRs were performed with Splink1 and SfU6-Rv 1 and Splink2 and SfU6-Rv2 as the primer pairs, respectively (primer sequences are provided in Table 2). The resulting amplimers were cloned into pGEM-T (Promega) and three independent clones were sequenced to determine the consensus.

TABLE 2
Primers used for splinkerette PCR.

| Primer | |
|---|---|
| name | Sequence (5′ to 3′) |
| Splink- | gatcccactagtgtcgacaccagtctctaatttttttttt |
| GATC-TOP | caaaaaaa |
| | (SEQ ID NO: 10) |
| Splink- | ctagccactagtgtcgacaccagtctctaatttttttttt |
| CTAG-TOP | caaaaaaa |
| | (SEQ ID NO: 11) |
| Splink- | tcgaccactagtgtcgacaccagtctctaatttttttttt |
| TCGA-TOP | caaaaaaa |
| | (SEQ ID NO: 12) |
| Splink- | agctccactagtgtcgacaccagtctctaatttttttttt |
| AGCT-TOP | caaaaaaa |
| | (SEQ ID NO: 13) |
| Splink- | cgaagagtaaccgttgctaggagagaccgtggctgaatga |
| bottom | gactggtgtcgacactagtgg |
| | (SEQ ID NO: 14) |
| Splink1 | cgaagagtaaccgttgctaggagagacc |
| | (SEQ ID NO: 15) |
| Splink2 | gtggctgaatgagactggtgtcgac |
| | (SEQ ID NO: 16) |
| SfU6-Rv1 | gcttcacgattttgcgtgtcatccttg |
| | (SEQ ID NO: 17) |
| SfU6-Rv2 | gggccatgctaatcttctctgtatcg |
| | (SEQ ID NO: 18) |

Genomic DNA Isolation and CEL-I nuclease assays. Genomic DNA was extracted from Sf9, HIGH FIVE™, BmN, and S2R+ cells using the WIZARD® genomic DNA extraction kit (Promega) according to the manufacturer's instructions. CEL-I nuclease assays were performed using known techniques. The primer sequences used to amplify various target loci are provided in Table 3.

TABLE 3
Primers used to amplify sequences surrounding
target sites.

| Primer | Target | |
|---|---|---|
| name | site | Sequence (5′ to 3′) |
| DmFDLsurv- | DmFDLt3 | acaggcctggtggtggtgtc |
| Fw | | (SEQ ID NO: 19) |
| DmFDLsurv- | DmFDLt3 | aaagttaagatccccggatttgagcac |
| Rv | | (SEQ ID NO: 20) |
| SfFDLt12- | SfFDLt1, | ggcagtttctaaccgcttacttttg |
| Fw | SfFDLt2 | (SEQ ID NO: 21) |
| SfFDLt12- | SfFDLt1, | cttactcgtagagagcgtgcagc |
| Rv | SfFDLt2 | (SEQ ID NO: 22) |
| SfFDLt3- | SfFDLt3 | cgcggacttctccttgacacag |
| Fw | | (SEQ ID NO: 23) |
| SfFDLt3- | SfFDLt3 | cgaacccgcagtccaggtac |
| Rv | | (SEQ ID NO: 24) |
| TnFDLsurv- | TnFDLt | atgaagtggtggggcga |
| Fw | | (SEQ ID NO: 25) |
| TnFDLsurv- | TnFDLt | gccacagctgtgtcgagtc |
| Rv | | (SEQ ID NO: 26) |
| BmFDLsurv- | BmFDLt1, | cttttatttatcgattcgggc |
| Fw | BmFDLt2, | (SEQ ID NO: 27) |
| | BmFDLt3 | |
| BmFDLsurv- | BmFDLt1, | gaatgcgctgtgatgtctac |
| Rv | BmFDLt2, | (SEQ ID NO: 28) |
| | BmFDLt3 | |

TIDE analysis. We performed TIDE analysis using a known technique. Briefly, we directly sequenced the PCR products amplified from genomic DNA extracted from Sf9 and various monoclonal SfFDLt1 isolates and used the sequencing results as queries for a TIDE web program (https://tide-calculator.nki.nl/). All analyses were performed with a default setting.

EGFP Reduction Assay. S2R-EGFP, Sf9-EGFP, Tn-EGFP, and BmN-EGFP cells were transfected with various CRISPR-Cas9 vectors targeting EGFP or a control vector encoding no sgRNA and selected for puromycin resistance. Puromycin-resistant survivors were analyzed using a GUAVA® easyCyte HT flow cytometer (Millipore) and EGFP positive cell populations were quantified using FlowJo software.

Expression and purification of hEPO. Two steps were used to isolate AcRMD2-p6.9-hEPO, a recombinant baculovirus encoding an N-terminally affinity-tagged version of hEPO. First, we recombined a gene encoding the Pseudomonas aeruginosa GDP-4-dehydro-6-deoxy-D-mannose reductase (rmd) cds under the control of the AcMNPV ie1 promoter into the chi-cth locus of a baculovirus vector called BacPAK6-p6.9-GUS to produce AcRMD2. Second, we recombined a honey bee melittin signal peptide, 8× HIS-tag, Strep II-tag, TEV recognition site, and mature hEPO cds under the control of the AcMNPV p6.9 promoter into the polh locus of AcRMD2. We used the resulting baculovirus to express and purify hEPO by known techniques.

Isolation and characterization of monoclonal SfFDLKO cell lines. Single cell clones were isolated from the polyclonal SfFDLt1 cell population, as described previously. Indels were analyzed by CEL-I nuclease assays and the Sf-fdl gene sequences in clones 4, 14, 32, and 49 were amplified, sequenced, and the sequences were analyzed by TIDE, as described previously.

Mass Spectrometry. N-glycans were enzymatically released from purified hEPO and derivatized using known methods, then analyzed by MALDI-TOF-MS using an Applied Biosystems SCIEX TOF/TOF 5800 (SCIEX), with 400 shots accumulated in reflectron positive ion mode. Structures were manually assigned to peaks based on knowledge of the insect cell N-glycan processing pathway. Quantification involved dividing the peak intensities of permethylated N-glycan structures by the total intensity of all annotated N-glycan peaks having >1% of total intensities.

### Certain Exemplary Embodiments

Example 1. Heterologous Insect U6 Promoters Fail to Support CRISPR-Cas9 Editing in Sf9 cells. When we undertook this effort, there were no known Sf or Tn RNA polymerase III promoters. However, as noted above, there were DmU6 and BmU6 promoters with the known ability to drive sgRNA expression in Dm and Bm cells, respectively (24-26). Thus, we chose to use the DmU6 and BmU6 promoters as potential surrogates for CRISPR-Cas9 genome editing in Sf9 and HIGH FIVE™ cells, based on their ability to drive sgRNA expression in other insect cell systems. Dm is a dipteran and Bm is a lepidopteran, so the former is relatively distantly and the latter more closely related to Sf and Tn, from which Sf9 and HIGH FIVE™ were derived.

We initially designed generic CRISPR-Cas9 vectors that included an Sf codon-optimized Streptococcus pyogenes (Sp) Cas9 coding sequence under the control of a baculovirus ie1 promoter, which provides constitutive transcription in a wide variety of organisms, followed by either the DmU6:96Ab or BmU6-2 promoter for sgRNA expression and a targeting sequence cloning site. These vectors also included a puromycin resistance marker (puromycin acetyl transferase, pac) under the control of baculovirus hr5 enhancer and ie1 promoter elements (depicted schematically in FIG. 1A). After constructing, mapping, and sequencing the generic DmU6:96Ab and BmU6-2 CRISPR-Cas9 vectors, we designed, synthesized, and inserted targeting sequences (shown in Table 1) for the Dm or Bm fdl genes (FIGS. 2A and 2C, respectively). We then examined the editing capacities of the products by transfecting Dm (S2R+) or Bm (BmN) cell lines, respectively, and performing CEL-I nuclease assays on puromycin resistant derivatives. The results of this control experiment showed the Dm-fdl gene was efficiently edited in S2R+ cells transfected with the DmU6 vector encoding the Dm-fdl-specific sgRNA and in S2R+ cells transfected with AcCas9DmFDLt3, a previously described CRISPR-Cas9 vector encoding a Dm-fdl-specific sgRNA, but not in S2R+ cells transfected with a vector encoding Cas9 alone (FIG. 2B). Similarly, the Bm-fdl gene was efficiently edited in BmN cells transfected with each of three BmU6-2 vectors encoding different Bm-fdl-specific sgRNAs, but not in BmN cells transfected with a vector encoding Cas9 alone (FIG. 2D). These results indicated our new CRISPR-Cas9 vectors produced functional Cas9 under ie1 promoter control, functional sgRNAs under DmU6:96Ab and BmU6-2 promoter control, and also showed they could be used for efficient CRISPR-Cas9 editing of endogenous gene targets in cells from the homologous species.

Therefore, we constructed DmU6:96Ab and BmU6-2 CRISPR-Cas9 vectors encoding sgRNAs with three different Sf-fdl targeting sequences (Table 1; FIG. 1B) and used them to transfect Sf9 cells in an effort to edit the Sf-fdl gene. However, CEL-I nuclease assays revealed no evidence of Sf-fdl indels in the resulting puromycin-resistant Sf9 derivatives (FIG. 1C). Because the results obtained with Dm and Bm cells indicated these vectors induced adequate Cas9 and pac expression, this result demonstrated the DmU6 and BmU6 promoters cannot support adequate sgRNA expression in Sf9 cells, which are derived from a heterologous insect species. Therefore, we concluded it was necessary to identify an endogenous SfU6 promoter to induce sgRNA expression in Sf9 cells.

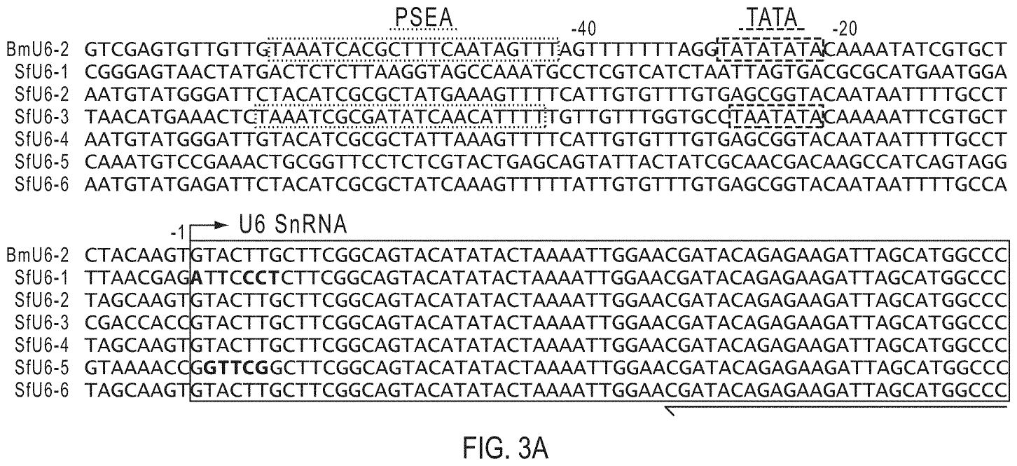

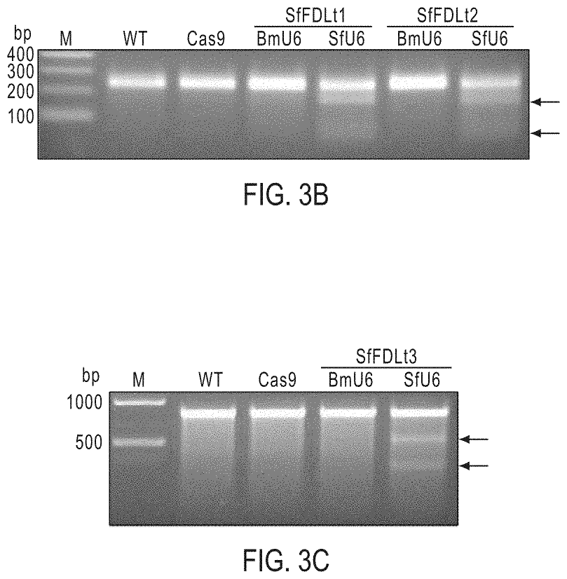

Example 2. A Newly Identified SfU6 Promoter Supports CRISPR-Cas9 Editing in Sf9 Cells. Using the BmU6-2 snRNA sequence as a query to search the Sf draft genome sequence, we found only one putative SfU6 snRNA coding sequence. We had no confidence in this hit because insect snRNA sequences are often derived from pseudogenes. Thus, we used splinkerette PCR to experimentally isolate SfU6 promoter candidates from Sf9 genomic DNA. This approach yielded six unique U6 snRNA upstream sequences (FIG. 3A; sequences BmU6-2, SfU6-1, SfU6-2, SfU6-3, SfU6-4, SfU6-5, and SfU6-6 correspond to SEQ ID NOs: 29-35, respectively) including the one (SfU6-3; SEQ ID NO: 32) identified using bioinformatics. Additional bioinformatics showed only SfU6-3 included the proximal sequence element A (PSEA; shown in dotted rectangles in FIG. 3A) and TATA box (shown in dashed rectangles in FIG. 3A) required for insect U6 promoter function. Sequences shown in FIG. 3A:

BmU6-2:
(SEQ ID NO: 29)
GTCGAGTGTTGTTGTAAATCACGCTTTCAATAGTTTAGTTTTTTTAGGTA
TATATACAAAATATCGTGCTCTACAAGTGTACTTGCTTCGGCAGTACATA
TACTAAAATTGGAACGATACAGAGAAGATTAGCATGGCCC
SfU6-1:
(SEQ ID NO: 30)
CGGGAGTAACTATGACTCTCTTAAGGTAGCCAAATGCCTCGTCATCTAAT
TAGTGACGCGCATGAATGGATTAACGAGATTCCCTCTTCGGCAGTACATA
TACTAAAATTGGAACGATACAGAGAAGATTAGCATGGCCC
SfU6-2:
(SEQ ID NO: 31)
AATGTATGGGATTCTACATCGCGCTATGAAAGTTTTCATTGTGTTTGTGA
GCGGTACAATAATTTTGCCTTAGCAAGTGTACTTGCTTCGGCAGTACATA
TACTAAAATTGGAACGATACAGAGAAGATTAGCATGGCCC
SfU6-3:
(SEQ ID NO: 32)
TAACATGAAACTCTAAATCGCGATATCAACATTTTTGTTGTTTGGTGCCT
AATATACAAAAATTCGTGCTCGACCACCGTACTTGCTTCGGCAGTACATA
TACTAAAATTGGAACGATACAGAGAAGATTAGCATGGCCC
SfU6-4:
(SEQ ID NO: 33)
AATGTATGGGATTGTACATCGCGCTATTAAAGTTTTCATTGTGTTTGTGA
GCGGTACAATAATTTTGCCTTAGCAAGTGTACTTGCTTCGGCAGTACATA
TACTAAAATTGGAACGATACAGAGAAGATTAGCATGGCCC
SfU6-5:
(SEQ ID NO: 34)
CAAATGTCCGAAACTGCGGTTCCTCTCGTACTGAGCAGTATTACTATCGC
AACGACAAGCCATCAGTAGGGTAAAACCGGTTCGGCTTCGGCAGTACATA
TACTAAAATTGGAACGATACAGAGAAGATTAGCATGGCCC
SfU6-6:
(SEQ ID NO: 35)
AATGTATGAGATTCTACATCGCGCTATCAAAGTTTTTATTGTGTTTGTGA
GCGGTACAATAATTTTGCCATAGCAAGTGTACTTGCTTCGGCAGTACATA
TACTAAAATTGGAACGATACAGAGAAGATTAGCATGGCCC

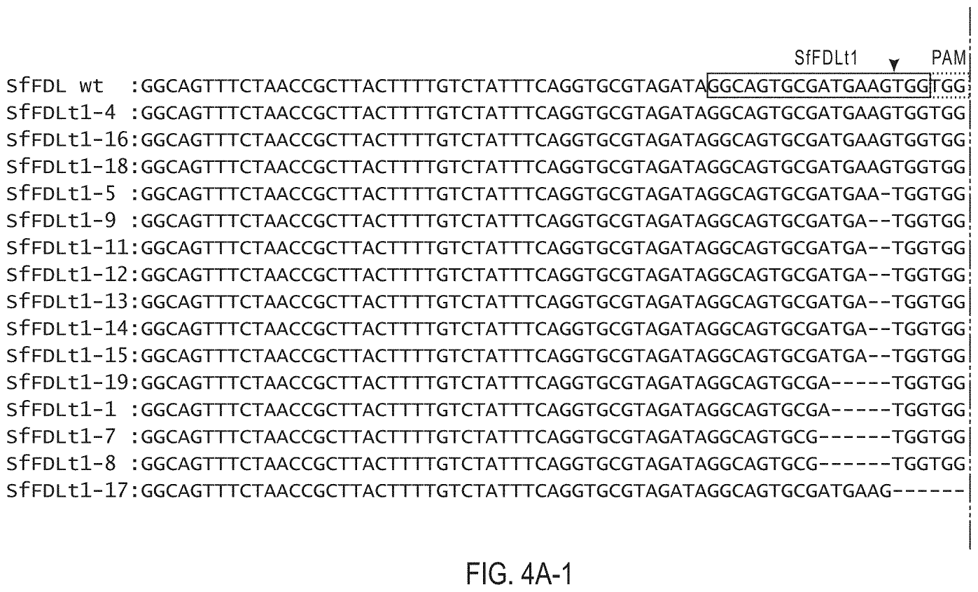

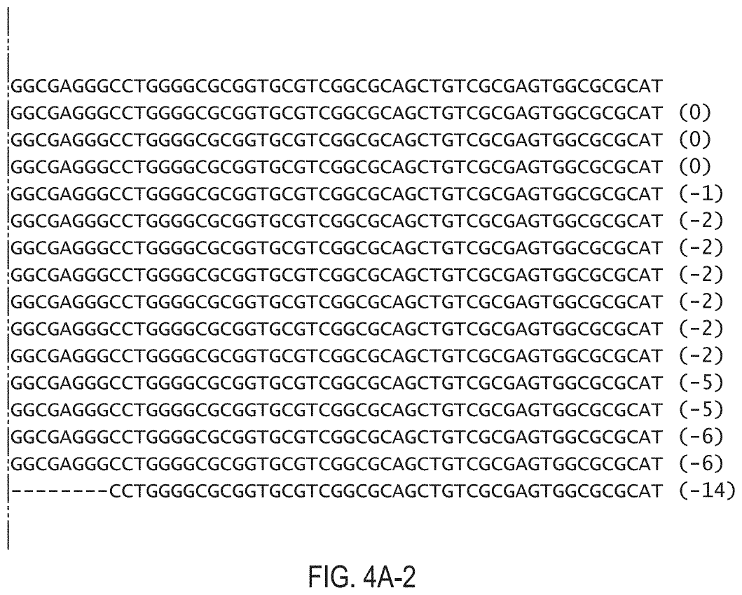

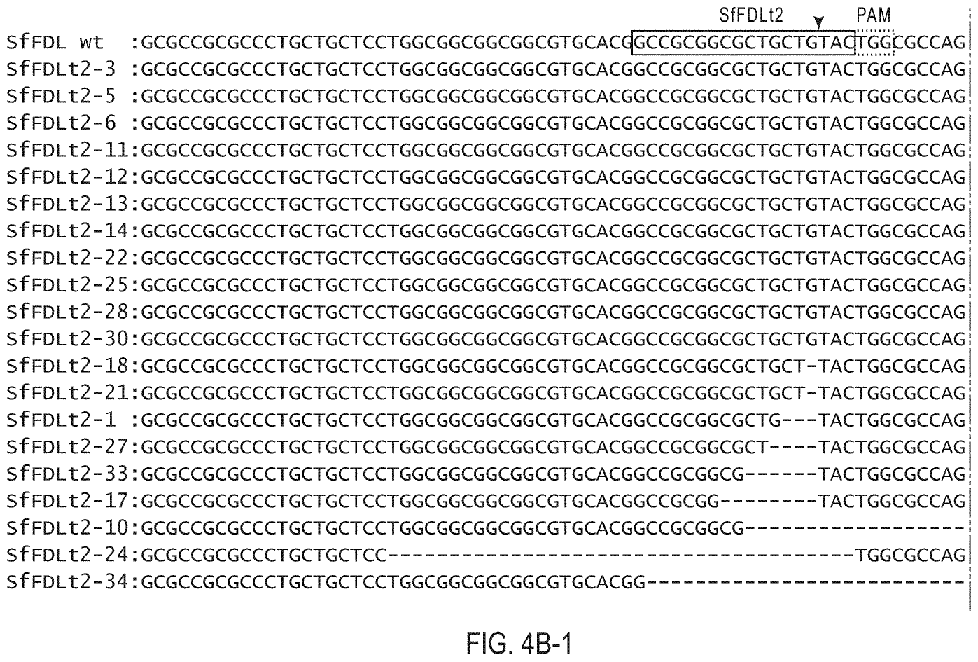

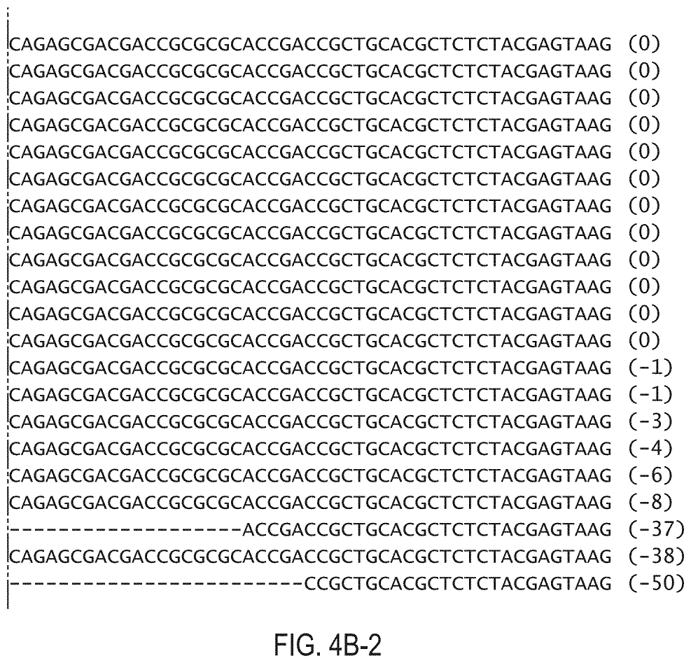

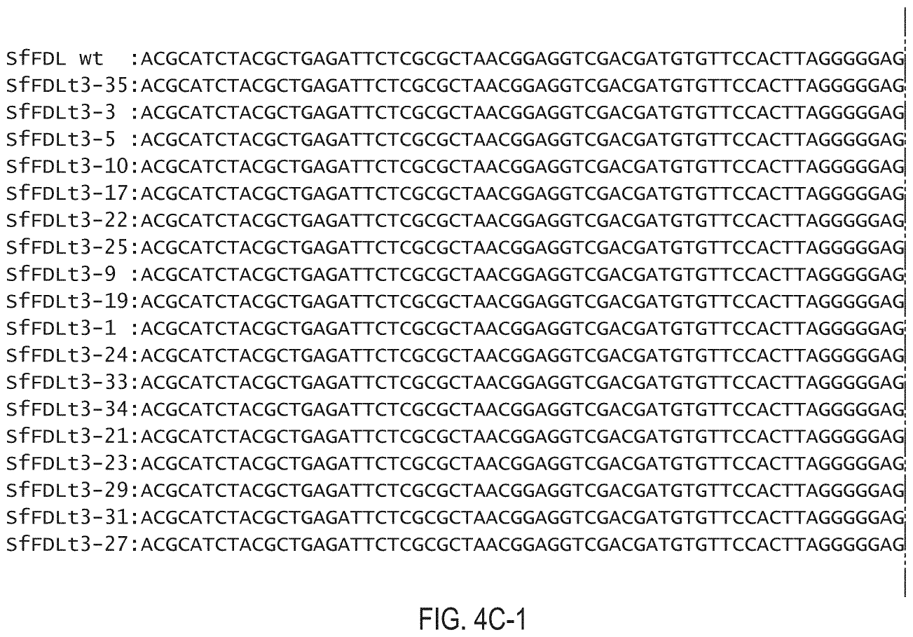

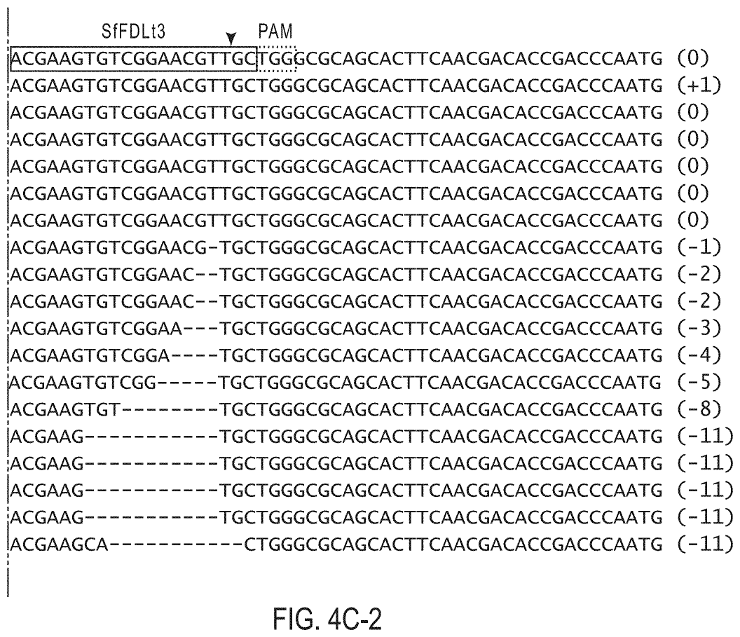

Based on these results, we used SfU6-3 (SEQ ID NO: 47) to construct a generic CRISPR-Cas9 vector (FIG. 1A) and then constructed three derivatives using the Sf-fdl targeting sequences previously inserted into the DmU6 and BmU6 CRISPR-Cas9 vectors (Table 1; FIG. 1B). We used each construct to transfect Sf9 cells, selected puromycin resistant derivatives, and then performed CEL-I nuclease assays with genomic DNAs from those cells. The results showed all three SfU6-3-based CRISPR-Cas9 vectors produced Sf-fdl indels (FIG. 3B) and this was confirmed by PCR and sequencing, as shown in FIGS. 4A-4C and Table 4. These results clearly demonstrated the SfU6-3 promoter, but not the DmU6:96Ab or BmU6-2 promoters, can be used for CRISPR-Cas9 editing in Sf9 cells.

TABLE 4
Indels found in SfFDLt1 monoclonal cell lines.

| | SfFDLt1 | SfFDLt1 | SfFDLt1 | SfFDLt1 |
|---|---|---|---|---|
| Indels | #4 | #14 | #32 | #49 |
| −1 bp | 2 | 1 | 5 | 1 |
| −2 bp | 8 | 3 | 2 | 8 |
| −6 bp | | 1 | | |
| −95 bp | | 3 | | |
| +76 bp | | | 3 | |

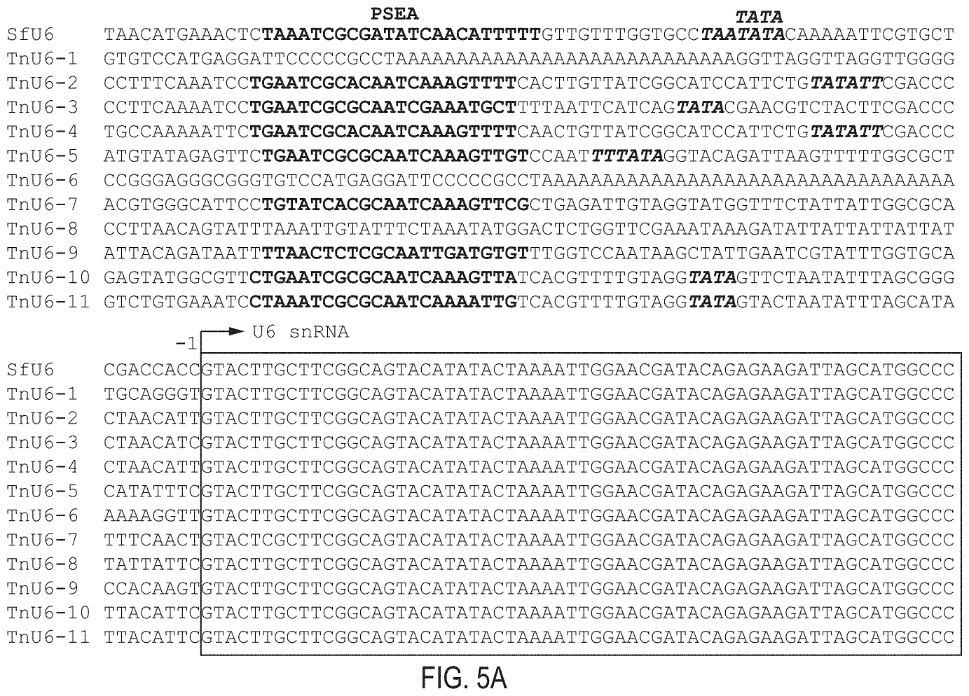

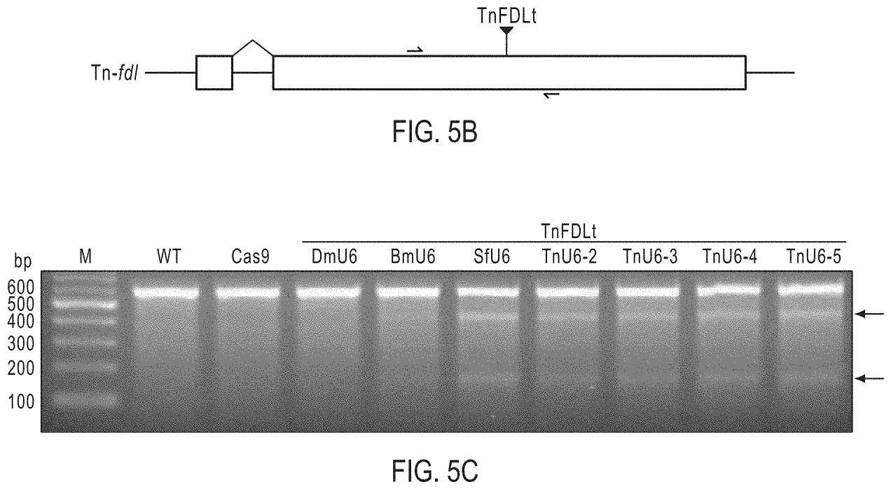

Example 3. Newly Identified TnU6 Promoters Support CRISPR-Cas9 Editing in Tn Cells. We extended these results by using splinkerette PCR to identify eight putative TnU6 promoters as potential tools for CRISPR-Cas9 editing of HIGH FIVE® cells (FIG. 5A). We then used TnU6-2, TnU6-3, TnU6-4, TnU6-5 (sequences shown below), all of which had PSEA and TATA elements, to construct generic CRISPR-Cas9 vectors. Finally, we inserted a Tn-fdl-specific targeting sequence (Table 1; FIG. 3B), transfected HIGH FIVE® cells with the resulting constructs, selected for puromycin resistance, and examined the cellular Tn-fdl genes using CEL-I nuclease assays. The results indicated the Tn-fdl gene was edited in each case, demonstrating TnU6-2, -3, -4, and -5 are all effective as promoters for CRISPR-Cas9 editing in HIGH FIVE® cells (FIG. 5C). Interestingly, the CEL-I nuclease assays also indicated the SfU6-3, BmU6-2, and DmU6:96Ab CRISPR-Cas9 vectors encoding the Tn-fdl-specific sgRNA produced efficient, inefficient, and no detectable Tn-fdl gene editing in HIGH FIVE® cells, respectively (FIG. 5C). These results showed the TnU6-2 (SEQ ID NO:49), TnU6-3 (SEQ ID NO:50), TnU6-4 (SEQ ID NO:51), TnU6-5 (SEQ ID NO:52) and SfU6-3 (SEQ ID NO:47) promoters can all be used for CRISPR-Cas9 editing in HIGH FIVE® cells.

TnU6-2:
(SEQ ID NO: 38)
CCTTTCAAATCCTGAATCGCACAATCAAAGTTTTCACTTGTTATCGGCAT
CCATTCTGTATATTCGACCCCTAACATTGTACTTGCTTCGGCAGTACATA
TACTAAAATTGGAACGATACAGAGAAGATTAGCATGGCCC
TnU6-3:
(SEQ ID NO: 39)
CCTTCAAAATCCTGAATCGCGCAATCGAAATGCTTTTAATTCATCAGTAT
ACGAACGTCTACTTCGACCCCTAACATCGTACTTGCTTCGGCAGTACATA
TACTAAAATTGGAACGATACAGAGAAGATTAGCATGGCCC
TnU6-4:
(SEQ ID NO: 40)
TGCCAAAAATTCTGAATCGCACAATCAAAGTTTTCAACTGTTATCGGCAT
CCATTCTGTATATTCGACCCCTAACATTGTACTTGCTTCGGCAGTACATA
TACTAAAATTGGAACGATACAGAGAAGATTAGCATGGCCC
TnU6-5:
(SEQ ID NO: 41)
ATGTATAGAGTTCTGAATCGCGCAATCAAAGTTGTCCAATTTTATAGGTA
CAGATTAAGTTTTTGGCGCTCATATTTCGTACTTGCTTCGGCAGTACATA
TACTAAAATTGGAACGATACAGAGAAGATTAGCATGGCCC

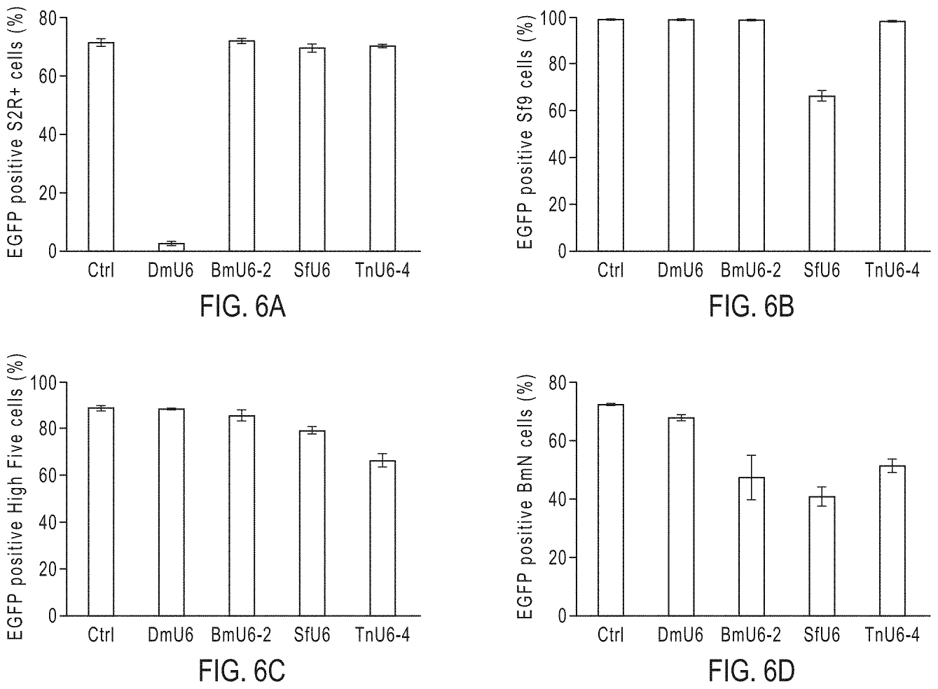

Example 4. CRISPR-Cas9 Editing Efficiencies Mediated by Various Insect U6 Promoters in various insect cell lines. Considering the U6 promoters derived from Tn and Sf both mediated Tn-fdl gene editing in HIGH FIVE™ cells, we chose to more quantitatively document the efficiencies of CRISPR-Cas9 editing provided by various insect U6 promoters in the various insect cell lines used in this study. First, we transformed S2R+, Sf9, HIGH FIVE®, and BmN cells with an EGFP expression plasmid. Then, we transfected each transformed derivative with CRISPR-Cas9 vectors encoding an EGFP-specific sgRNA under the control of Dm, Bm, Sf, or Tn U6 promoters and measured cellular fluorescence. The results showed only the homologous U6 CRISPR-Cas9 vectors significantly reduced fluorescence in S2R+ and Sf9 cells (FIGS. 6A and B), whereas the U6 promoters from several species reduced fluorescence in Tn and Bm cells (FIGS. 6C and D). Overall, among those tested, the DmU6:96Ab (underlined in SEQ ID NO:46, bp 7201-7600), SfU6-3 (SEQ ID NO:47), and TnU6-4 (SEQ ID NO:51) promoters would be the best choices for CRISPR-Cas9 editing in S2R+, Sf9, and HIGH FIVE® cells, respectively. In contrast, the BmU6-2 (SEQ ID NO:48), SfU6-3 (SEQ ID NO:47), and TnU6-4 (SEQ ID NO:51) promoters all provided about the same efficiencies and the heterologous SfU6-3 promoter would likely be the best choice for CRISPR-Cas9 editing in BmN cells.

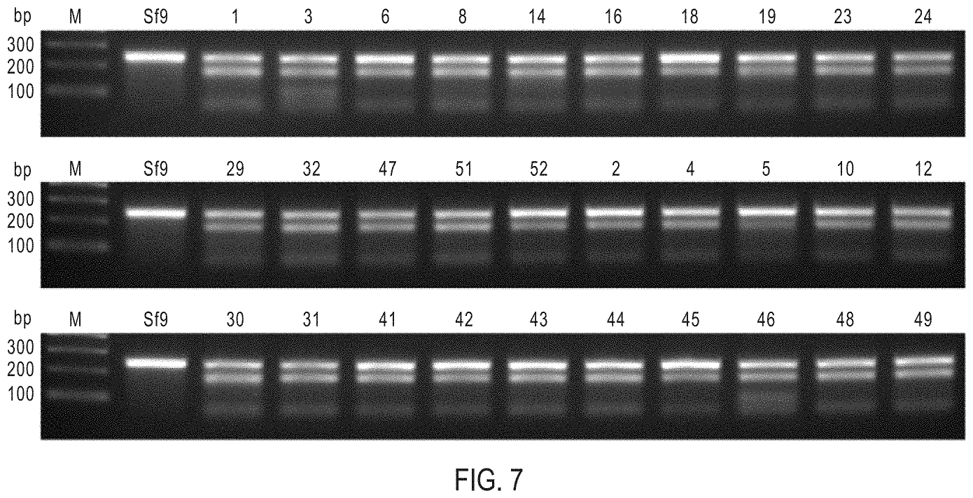

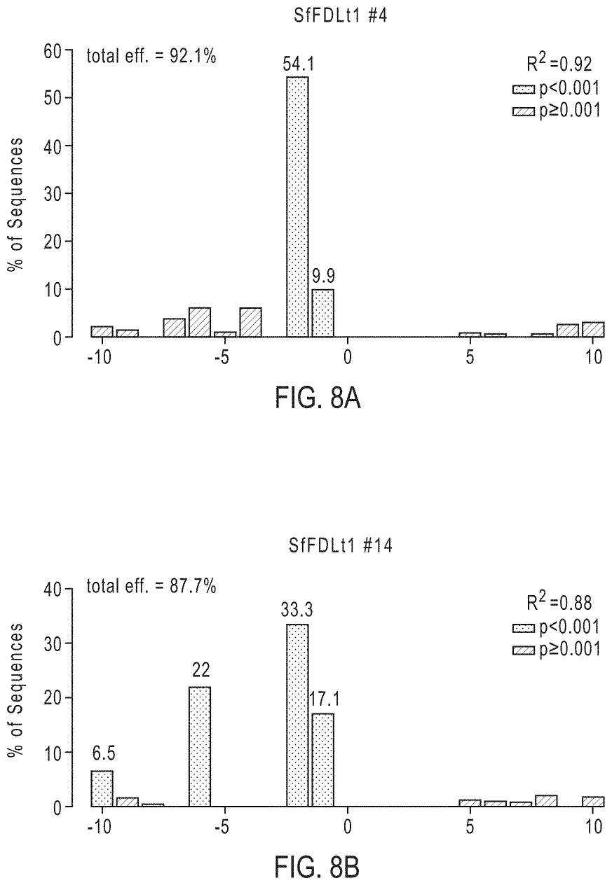

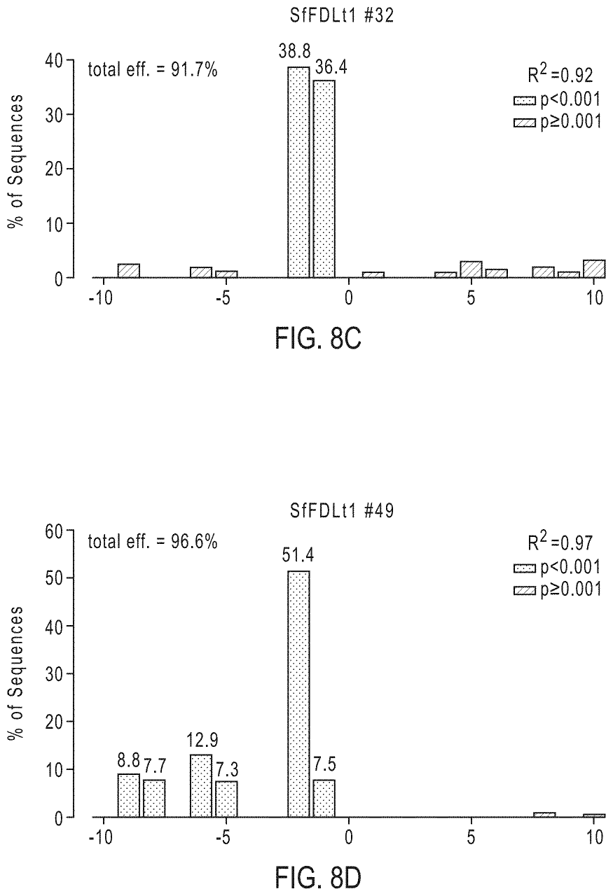

Example 5. Phenotypic Impact of Gene Editing with SfU6-3-SfFDLt1 CRISPR-Cas9 Vector in Sf9 Cells. Subsequently, we assessed the phenotypic impact of gene editing using one of the new CRISPR-Cas9 tools created in this study. Sf9 cells were transfected with the CRISPR-Cas9 vector encoding the Sf-FDLt1 sgRNA under SfU6-3 promoter control, puromycin-selected, and the resulting polyclonal cell population (SfFDLt1) was used to isolate 30 single cell clones. The Sf-fdl sequences in the parental Sf9, polyclonal SfFDLt1, and SfFDLt1 clones were then examined by CEL-I nuclease assays and TIDE analysis, as described above. The CEL-I nuclease assay results indicated all 30 clones had Sf-fdl indels (FIG. 7) and TIDE analysis revealed four clones had no wild-type Sf-fdl sequences or potentially functional in-frame deletions (FIG. 8; Tables 4 and 5).

TABLE 5
Proportions of wild type (WT) and in-frame (−3 bp)
deletions in monoclonal SfFDLt1 cell lines as determined
using the TIDE program. Four clones with no wild-type
sequences or potentially functional in-frame deletions
were identified (clones 4, 14, 32, and 49, shown in bold
and italicized text).

| Clone # | WT (%) | −3 bp (%) |
|---|---|---|
| 1 | 0 | 26.8 |
| 2 | 0 | 22.3 |
| 3 | 0.7 | 0 |
| [symbol] | [symbol] | [symbol] |
| 5 | 56.8 | 1.1 |
| 6 | 0.1 | 23.2 |
| 8 | 1.4 | 42.1 |
| 10 | 0 | 5.5 |
| 12 | 1.7 | 10.1 |
| [symbol] | [symbol] | [symbol] |
| 18 | 0 | 6 |
| 23 | 0 | 8.9 |
| 24 | 0 | 1.7 |
| 29 | 27 | 0 |
| 30 | 1.3 | 1 |
| 31 | 2.1 | 14.3 |
| [symbol] | [symbol] | [symbol] |
| 41 | 1.1 | 12.2 |
| 42 | 0 | 11.7 |
| 43 | 0 | 21.7 |
| 44 | 0 | 6.2 |
| 45 | 0.5 | 0 |
| 46 | 0 | 17.5 |
| 47 | 0 | 8.5 |
| 48 | 8.5 | 14.8 |
| [symbol] | [symbol] | [symbol] |
| 51 | 1 | 8.9 |
| 52 | 0 | 6.7 |

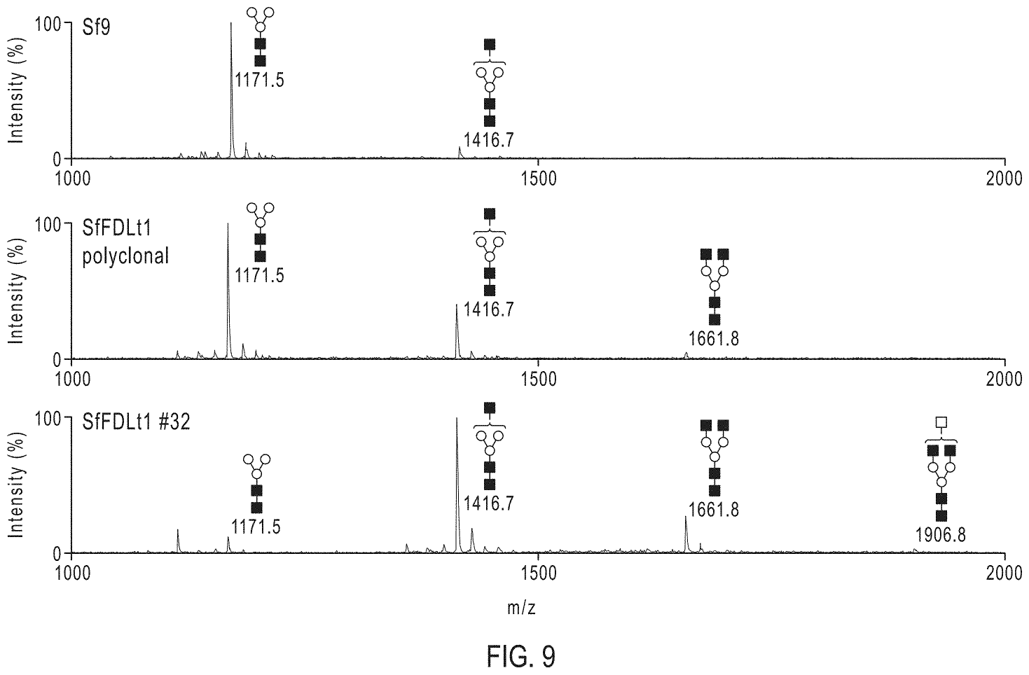

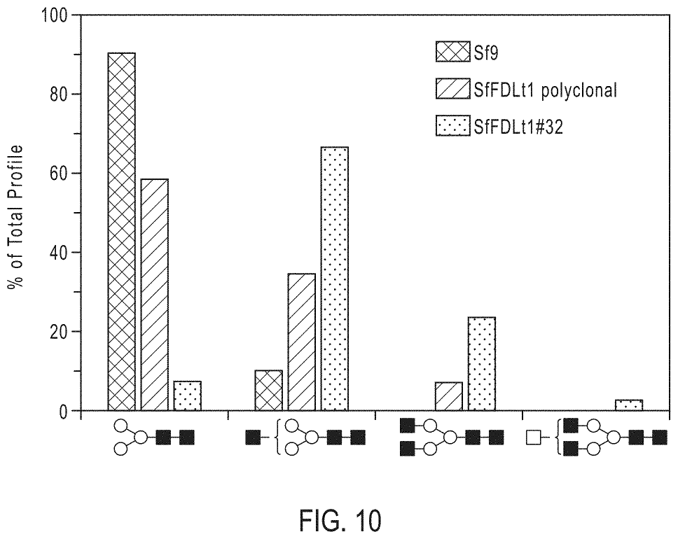

We subsequently infected one of those clones (#32), as well as Sf9 cells and the polyclonal SfFDLt1 cell population, with a recombinant baculovirus encoding an affinity-tagged version of human erythropoietin (hEPO) and purified the secreted product from each culture, as described. We then enzymatically released the N-glycans from each purified protein preparation and analyzed the permethylated glycan structures by MALDI-TOF-MS, as described. The spectra showed the major N-glycan on hEPO from Sf9 and SfFDLt1 (polyclonal) cells was Man₃GlcNAc₂, whereas the major N-glycan on hEPO from SfFDLt1 #32 was GlcNAcMan₃GlcNAc₂ (FIG. 9). A quantitative analysis showed Man₃GlcNAc₂ represented about 90%, 60%, and 8% of the total N-glycans on hEPO from Sf9, SfFDLt1 (polyclonal), and SfFDLt1 clone #32, respectively, as shown in FIG. 10. Reciprocally, GlcNAcMan₃GlcNAc₂ represented about 10%, 30%, and 65% of total N-glycans on hEPO from Sf9, SfFDLt1 (polyclonal), and SfFDLt1 clone #32, respectively (FIG. 10). Finally, GlcNAc₂Man₃GlcNAc₂ was only detected on hEPO from SfFDLt1 (polyclonal), and SfFDLt1 #32 (FIG. 10).

These results clearly demonstrate the phenotypic impact of genome editing with the SfU6-3-SfFDLt1 CRISPR-Cas9 vector in Sf9 cells. Specifically, the structures of the N-glycans observed in the Sf9 cells treated with this vector reveal a partial (polyclonal) and nearly complete (clone #32) loss of FDL function resulting from fdl editing with this vector (FIGS. 9 and 10).

We conclude that the novel CRISPR-Cas tools disclosed herein can be used to engineer host pathways in efforts to enhance and expand the capabilities of the BICS. These tools will enable far more sophisticated host-cell engineering efforts, which to date, have been limited to using non-homologous recombination to knock-in genes at random sites in the insect cell genome. Thus, these new tools will enable new efforts to enhance and expand the utility of the BICS as a recombinant protein production platform.

We initially tested Dm and Bm U6 promoters that were previously shown to direct effective sgRNA expression and CRISPR-Cas9 mediated genome editing in dipteran and lepidopteran insect cells. We assumed these promoters might drive these same functions in Sf and Tn cells, which would have allowed us to quickly produce CRISPR-Cas9 vectors for the BICS.

In fact, CRISPR-Cas9 vectors encoding Dm- or Bm-fdl-specific targeting sequences under DmU6 or BmU6 promoter control produced indels in cell lines from homologous species (FIGS. 2B-C). However, CRISPR-Cas9 vectors with these same Dm or Bm U6 promoters encoding sgRNAs with Sf- or Tn-fdl-specific targeting sequences failed to produce any detectable indels in Sf (FIGS. 1C-D) or Tn cells (FIG. 5C), respectively. This forced us to identify putative Sf and Tn U6 promoters, which we then used to produce CRISPR-Cas9 vectors encoding sgRNAs with the same Sf- or Tn-fdl-specific targeting sequences. We found CRISPR-Cas9 vectors with the homologous U6 promoters efficiently produced indels in Sf (FIGS. 3B and 3C) and Tn (FIG. 5C) cells, respectively.

We subsequently established an EGFP reduction assay, which could be used to more quantitatively measure the relative efficiencies of editing by CRISPR-Cas9 vectors encoding a GFP-specific sgRNA under the control of various insect U6 promoters in different insect cell species. The results indicated only the CRISPR-Cas9 vectors with homologous U6 promoters significantly reduced GFP expression in Dm and Sf cells (FIGS. 6A-D). In contrast, while the homologous U6 promoter provided the highest CRISPR-Cas9 editing efficiency in HIGH FIVE™ cells, SfU6-3 also provided a reasonable efficiency and the Bm, Sf, and Tn promoters all provided about the same efficiencies of CRISPR-Cas9 editing in BmN cells. These results indicate SfU6-3 has the broadest, while DmU6:96Ab has the narrowest host range among the insect U6 promoters tested in the insect cell lines we tested.

It was previously shown that a recombinant baculovirus designed to express Cas9 and sgRNAs under the control of mammalian promoters was capable of inducing genomic editing when the vector was transduced in mammalian cells. In contrast, we have created new CRISPR-Cas9 tools designed to express Cas9 and sgRNAs under the control of baculovirus and insect cell promoters. The utility of these novel constructs have been demonstrated by their ability to induce genome editing in the BICS.

Our results demonstrate that our novel constructs can be used for host cell engineering in the BICS. As disclosed herein, we targeted fdl, which encodes a key enzyme that distinguishes insect and mammalian cell protein N-glycosylation pathways by antagonizing N-glycan elongation. As such, fdl has been a high priority target for knockout, as this would facilitate efforts to glycoengineer the BICS and other insect-based recombinant protein production platforms for high efficiency mammalian-type protein N-glycosylation. It has been demonstrated various RNAi approaches can reduce FDL activity, but with little or no phenotypic impact on N-glycan processing. We previously used existing CRISPR-Cas9 tools to knockout Dm fdl in S2R+0 cells and demonstrate this had the expected impact on N-glycan processing. However, we were unable to knockout Sf-fdl or Tn fdl until we created the tools needed for site-specific gene editing in the BICS. We then used a CRISPR-Cas9 vector encoding a Sf-fdl-specific sgRNA under the control of the SfU6-3 promoter to produce polyclonal and monoclonal Sf9 cell derivatives. CEL-I nuclease assays and TIDE analysis indicated this CRISPR-Cas9 vector directed efficient editing of the Sf-fdl gene (FIGS. 8A-D; Table 2). Finally, we documented the phenotypic impact of these genotypic changes by analyzing the N-glycans isolated from recombinant hEPO produced by polyclonal and monoclonal Sf-fdl knockout cells described in this study. As expected, we observed reduced proportions (<10% of total) of paucimannose (Man₃GlcNAc₂) and increased proportions (>65% of total) of terminally GlcNAcylated (GlcNAc₁₋₂Man₃GlcNAc₂) structures on hEPO produced by SfFDLt1 cells, as compared to Sf9 cells (FIGS. 9 and 10). Thus, we have clearly demonstrated that the novel CRISPR-Cas9 vectors of the current teachings are useful for site-specific genome editing and that they can be used successfully for host cell engineering in the BICS.

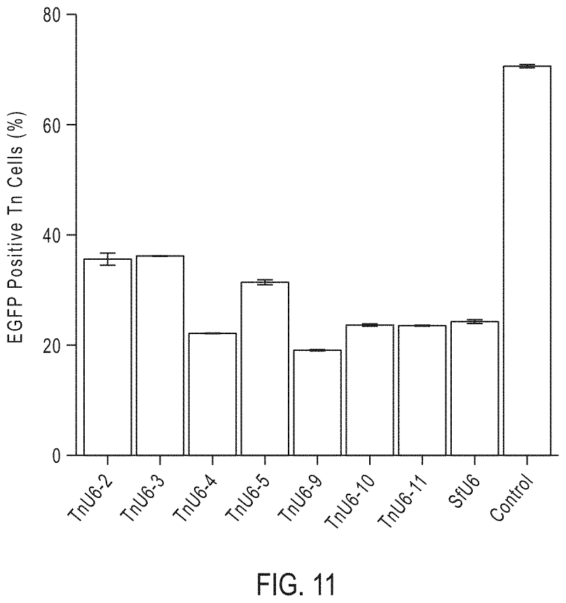

Example 6. Three additional TnU6 Promoters Support CRISPR-Cas9 Editing in Tn Cells. Finally, we extended our initial quantitative analysis of the CRISPR-Cas9 editing efficiencies provided by TnU6 promoters (FIG. 6) to include three additional putative TnU6 promoters identified by bioinformatic analysis of the Tn genome. Briefly, we constructed CRISPR-Cas9 vectors analogous to those shown in FIG. 1A, in which the U6 promoter was TnU6-9, TnU6-10, or TnU6-11 (FIG. 5A) and the sgRNA was specifically targeted to the EGFP gene. We then used the resulting plasmids to transfect High Five®-EGFP cells, selected for puromycin resistance, and measured cellular fluorescence, as described for the experiment shown in FIG. 6. The results showed transformation with CRISPR-Cas9 vectors containing any of the seven TnU6 promoters reduced High Five®-EGFP cell fluorescence to about 50% of control levels (FIG. 11). The results also showed the CRISPR-Cas9 vectors with the TnU6-4, -9, -10, and -11 promoters (SEQ ID NOs: 51, 53, 54, and 55) provided the most efficient CRISPR-Cas9 editing, whereas those with the TnU6-2, -3, and -5 promoters (SEQ ID NOs: 38, 39, and 41) provided somewhat less efficient editing (FIG. 11). Overall, these results show the TnU6-4, TnU6-9, TnU6-10, or TnU6-11 promoters (SEQ ID NOs: 51, 53, 54, and 55) and even the SfU6-3 promoter (SEQ ID NO: 47) can drive CRISPR-Cas9 editing at about the same efficiencies in High Five™ cells.

TnU6-9:
(SEQ ID NO: 53)
CAATAAATTAATGCCTAAAGTGCTTTCGTCGTACATTTTGATGTTAGAAA
ACACTCATATTAGGGAATACTTTTTTACATATGGCAACTGTCTTAAAAAA
AGTGTAAAAGCAAGAAATGAATGTGCGATTTGTTATTTTTTATGTTTATA
TTGATAAATAAATAATAAAAATGTGTCTCGTATGCGTGGAAATGGACATT
AAGGGAAAAAAAAAAACAATTGGTGGAATTATTTATATACAGAACCAGAT
GATTTAAGTGTTTATAATCAGTAAAACCAAGCGTGGATTATAGATTTTTA
TATTTACTAAACAAAATTGTTATAAAAATAAGTAATATTTGGAAAATAAA
GTAAAGTGCTCCGGTTAACAACATTGTACATTTTGTTTTGATAGTGCAAT
TATAAATTCTGTAATGGCAAATCATTGGAAATATCTTGTACCATAGAAAT
AAATTATGTATTTAAAACGAATGTCTATTTTATTCAGCTGAGGGCGTAAT
CTGGCCTTCGTTGTCGTGATAAGAGTCGCATCAGAATTAAATATAAATCA
ATTGTTAAAACTTGTTTGTTTTTACCTAGATTTAACTTAATAATGCATTT
ATTAAAAATAGCAAAACAACAGCCATCCGAGTTTCTGCTGTACTCTCAAA
TAGGTAACTGGCTAAGATGTGATTGAGAACAGCGCCATCTACATTCTTCG
GATTTAAACTCTTGGTGCGCTAGTTCGCATGTGTTTTCGTTAGTTCATAC
CTGTGACACAGATGTCGCTAGTGTGGCAAGATTAAAACAATTTGCTTATT
TTCTCTTATAAATCAATTAAATGAGAGAGAAATGAAACTTTCTGACCCAA
AGATTGAATAATTAATTATTGTTTTACAAATATATGTAAGTTTTTTTTTT
AAATGTAAGTAATTCTTAAGTTATTACAGATAATTTTAACTCTCGCAATT
GATGTGTTTGGTCCAATAAGCTATTGAATCGTATTTGGTGCACCACAAGT
TnU6-10:
(SEQ ID NO.: 54)
GATAATGAAAACTTTTGCTATAGGACTCATGCCTTATCTGATAATAAGAA
CAATTTAATCTACAAAACAAAATCAATCCTGTAAGTAAAGAAAAATAGTT
TTTCAAGATTGTCAATAGATGGCGCTGTTACAACCTTCCTTACATTTAGA
GATGCAGTTTCAAATTAAAAGAAAATACAATTTACCGTGTTTAATAGCAA
ATAATACAAATATTTGTTGTTTCGAACTATTTAATCTATTGATAAATTGT
CTTCAGTGATATCAGATAAGATATCGTTTAAAGTAGGTACACAATCTTGT
AAATATCATTATCTAACAGATGTTTTAAATTGTTGCTATATTTATGATGA
ATCATATTATGATCGCCGATAGTTGCTAACACACCCGCAACCCCATTCCG
TACGGGGTTTCTTACGATTTAAAAATTCAGTACAGGACGGAACACTCAAA
ATGCTTTAAAGAATACGAGTAATGACAAATTTAATCCAGGTGCCATTCAT
TCAATGGCCATTGCAAATAGAGAACCTGATTGAGGCCCAGCCCAGGCTTA
TGATACTTATACTTACCTTCAATACGCAATAATAGCAGTTTGTGTACACC
TAAAAGACAAATTCTTTTTTTATAAAACGCTTTTTTTCGAAATCTCATTG
AAACATTGCTTTGGGACGCAATTGATAGTACACCATATAAAAACATTGGT
CCATCACAGAACATTGTGAATAGAAATAGCTTTGTGTGTTGACATATTTT
AAATAAATATTTGTATGTTTTAGTTGATAATTAAAATTTACAACTATCCT
GAATGGTTAGTTTTCATCTCTGCAATGCATTGTAGCGACCGTGGCTGCAT
AAAAAAAATATTTAGTTTTTTTATATTGATCGATAGATGGCGCTGTTACA
AGCTTCCGAACATCAGTCGTTGGAGTATGGCGTTCTGAATCGCGCAATCA
AAGTTATCACGTTTTGTAGGTATAGTTCTAATATTTAGCGGGTTACATTC
TnU6-11:
(SEQ ID NO.: 55)
TAAGTAAACAAAAATATCATTTTATTTTCTTTAAAGTTAAATTTTATTAA
TTTACAAGAAGTACTGTTTTCATAAAATTTTTAAGTTTGGGTAGATTTTT
CTATTATTATTTGAATTTATTTGTAATGAGGCTGATCATTTATTAACCTT
CAGAGATCTTTATAATAATGGGTGTATTTTCAACTAAATAAAATACAAAA
AGAAAAATTTAACAAAATAGAAATGAGAAAAGGTATTTTTATTATTTTTC
ACTTAAAATAAAACTTGAATATTGTTACATAATTTTGGAATTGCTAAAAT
AAAAACAAAAGTCCTATTTTTAATATGTAAGTTAAGGTCTCTGCTAGGTA
TTATTGAAATGTTCACTCTCATAAACATAGTTTTTAAATAAATACATGTA
GGTAAAATATACGAATTTATATATTAAGTAAAAAATACTTTTGTAAATGT
CTTAAACTGCCAAGGGCTATTGATTTTAGGAAAATTAGTTCCAATAATTC
AACTAAAATTATTAAAACTAAAGTATTTTTACAATTTAGAAAAATTAAAA
TAAAATAATTTTAGTTAAAAACGGATTCCGTCCATAATTTATTTTTAGAT
AAACATTTGTAAATATTTTTCGTTTTTTTTTTATATGAAATGCATTTATT
AATTTTATTTAATTAGCCATAATAATGTGACAGAGTTAATCTGGATTCTG
ATTTTATGAATTTATTAAATCATTTTAAAAAGTTATTTGTATTTTGATTC
CATCTCTTAATTACTCATTTAGTAATTTATTAATATTAAATCGTAATTAT
TTAACTACACTGCAAAATTTTAATACCGCATCCATGTGTTCTACCTTTAC
TCAATGTTGTGGTAATTACTAGATTCGTCGATAGATGGCGCTGTGACTGC
TCCTCATACATTGTAAGCGTTGTCTGTGAAATCCTAAATCGCGCAATCAA
AATTGTCACGTTTTGTAGGTATAGTACTAATATTTAGCATATTACATTC

### Certain Exemplary Kits

In certain embodiments, kits are provided to expedite the performance of various disclosed DNA vectors and methods for using such vectors. Kits serve to expedite the performance of certain method embodiments by assembling two or more reagents and/or components used in carrying out certain methods. Kits may contain reagents in pre-measured unit amounts to minimize the need for measurements by end-users. Kits may also include instructions for performing one or more of the disclosed methods. In certain embodiments, at least some of the kit components are optimized to perform in conjunction with each other. Typically, kit reagents may be provided in solid, liquid, or gel form.

Certain kit embodiments comprise at least one DNA vector of the current teachings, and cells derived from a lepidopteran insect. In certain embodiments, the DNA vector comprises a Streptococcus pyogenes Cas9 (SpCas9) coding sequence operably linked to a first transcriptional control element; a single guide RNA (sgRNA) expression cassette comprising a targeting sequence cloning site and a sgRNA coding sequence operably linked to a second transcriptional control element; and a selectable marker operably linked to a third transcriptional control element. In certain embodiments, the DNA vector comprises a lepidopteran U6 promoter. In certain kit embodiments, the U6 promoter comprises comprises SEQ ID NO: 47; and the lepidopteran insect cells are derived from Spodoptera frugiperda, Trichoplusia ni, or Bombyx mori. In certain kit embodiments, the U6 promoter comprises SEQ ID NO: 51 and the lepidopteran insect cells are derived from Trichoplusia ni. According to certain embodiments, kits the U6 promoter comprises SEQ ID NO: 53, SEQ ID NO: 54, or SEQ ID NO: 55 and the lepidopteran insect cells are derived from Trichoplusia ni. In certain kit embodiments, the U6 promoter comprises SEQ ID NO: 47, SEQ ID NO: 48, or SEQ ID NO: 51; and wherein the lepidopteran insect cells are derived from Bombyx mori.

Although the disclosed teachings have been described with reference to various applications, constructs and vectors, it will be appreciated that various changes and modifications may be made without departing from the teachings herein. The foregoing examples are provided to better illustrate the present teachings and are not intended to limit the scope of the teachings herein. Furthermore, various presently unforeseen or unanticipated alternatives, modifications, variations or improvements therein may be subsequently made by those skilled in the art which are also intended to be encompassed by the following claims. Certain aspects of the present teachings may be further understood in light of the following claims.

1. A DNA vector comprising:
a Streptococcus pyogenes Cas9 (SpCas9) coding sequence operably linked to a first transcriptional control element;
a single guide RNA (sgRNA) expression cassette comprising a targeting sequence cloning site and a sgRNA coding sequence operably linked to a second transcriptional control element; and
a selectable marker operably linked to a third transcriptional control element.

2. The DNA vector of claim 1, wherein the first transcriptional control element comprises a baculovirus immediate early promoter, a baculovirus early promoter, a baculovirus enhancer, a polyadenylation signal, or combinations thereof.

3. The DNA vector of claim 2, wherein the first transcriptional control element comprises a baculovirus ie1 promoter, a baculovirus ie2 promoter, a baculovirus ie0 promoter, a baculovirus etl promoter, a baculovirus gp64 promoter, a baculovirus hr1 enhancer, a baculovirus hr2 enhancer, a baculovirus hr3 enhancer, a baculovirus hr4 enhancer, a baculovirus hr5 enhancer, a p10 polyadenylation signal, or combinations thereof.

4. The DNA vector of claim 1, wherein the second transcriptional control element comprises a lepidopteran insect cell promoter.

5. The DNA vector of claim 4, wherein the lepidopteran insect cell promoter is a lepidopteran U6 promoter.

6. The DNA vector of claim 5, wherein the lepidopteran U6 promoter is derived from Spodoptera frugiperda.

7. The DNA vector of claim 6, wherein the Spodoptera frugiperda U6 promoter comprises SEQ ID NO: 47.

8. The DNA vector of claim 5, wherein the lepidopteran insect U6 promoter is derived from Trichoplusia ni.

9. The DNA vector of claim 8, wherein the Trichoplusia ni U6 promoter is SEQ ID NO: 51.

10. The DNA vector of claim 8, wherein the Trichoplusia ni U6 promoter is SEQ ID NO: 53, SEQ ID NO:54, or SEQ ID NO:55.

11. The DNA vector of claim 2, wherein the targeting sequence cloning site comprises two adjacent type IIS restriction endonuclease sites.

12. The DNA vector of claim 11, wherein the targeting sequence cloning site comprises at least one SapI recognition site.

13. The DNA vector of claim 2, wherein the sgRNA coding sequence comprises SEQ ID NO: 45.

14. The DNA vector of claim 1, wherein the selectable marker comprises a puromycin, a blasticidin S, a G418, a hygromycin, a zeocin, or a nourseothricin resistance marker.

15. The DNA vector of claim 1, wherein the third transcriptional control element comprises a baculovirus promoter, a Respiratory Syncytial Virus (RSV) promoter, a copia promoter, a gypsy promoter, a piggyBac promoter, a cytomegalovirus immediate early promoter, a baculovirus enhancer, a baculovirus p10 polyadenylation signal, or combinations thereof.

16. The DNA vector of claim 15, wherein the baculovirus promoter comprises a baculovirus ie1 promoter, a baculovirus ie2 promoter, a baculovirus ie0 promoter, a baculovirus etl promoter, or a baculovirus gp64 promoter; and wherein the baculovirus enhancer comprises a baculovirus hr1 enhancer, a baculovirus hr2 enhancer, a baculovirus hr3 enhancer, a baculovirus hr4 enhancer, or a baculovirus hr5 enhancer.

17. The DNA vector of claim 16, wherein the third transcriptional control element comprises a baculovirus ie1 promotor, a baculovirus hr5 enhancer, and p10 polyadenylation signal.

18. The DNA vector of claim 1, wherein the SpCas9 coding sequence is codon optimized for Spodoptera frugiperda, the selectable marker is codon optimized for Spodoptera frugiperda, or both the SpCas9 coding sequence and the selectable marker are codon optimized for Spodoptera frugiperda.

19. The DNA vector of claim 1, wherein the SpCas9 coding sequence is codon optimized for Spodoptera frugiperda and the first transcriptional control element comprises a baculovirus ie1 promoter and a p10 polyadenylation signal; wherein the sgRNA coding sequence comprises SEQ ID NO: 45 and the second transcriptional control element comprises a lepidopteran U6 promoter; and wherein the selectable marker is codon optimized for Spodoptera frugiperda and encodes a puromycin acetyl transferase and the third transcriptional control element comprises a baculovirus ie1 promotor and a baculovirus hr5 enhancer.

20. An insect cell transformed with the DNA vector of claim 4, wherein the DNA vector further comprises a targeting sequence inserted in the targeting sequence insertion site and operably linked to a second transcriptional control element; and wherein the insect cell is derived from Spodoptera frugiperda, Trichoplusia ni or Bombyx mori.

21. The insect cell of claim 20, wherein the insect cell is derived from Sf-RVN cells, Sf9 cells, Sf21 cells, EXPRESSF+® cells, SUPER 9® cells, Tn-NVN cells, Tn368 cells, HIGH FIVE® cells, TNI PRO® cells, Ea4 cells, BTI-Tnao38 cells, or BmN cells.

22. An insect cell transformed with the DNA vector of claim 19, wherein the DNA vector further comprises a targeting sequence inserted in the targeting sequence insertion site and operably linked to the second transcriptional control element; and wherein the insect cell is derived from Spodoptera frugiperda, Trichoplusia ni or Bombyx mori.

23. The insect cell of claim 22, wherein the insect cell is derived from Sf-RVN cells, Sf9 cells, Sf21 cells, EXPRESSF+® cells, SUPER 9® cells, Tn-NVN cells, Tn368 cells, HIGH FIVE® cells, TNI PRO® cells, Ea4 cells, BTI-Tnao38 cells, or BmN cells.

24. A method for obtaining a modified lepidopteran cell comprising a newly-introduced genome editing function resulting in a modified cellular phenotype, the method comprising:
transfecting a lepidopteran insect cell with the DNA vector of claim 4, wherein the vector further comprises SEQ ID NO:2 inserted in the targeting sequence cloning site and operably linked to a second transcriptional control element;
incubating the transfected cells in a selective growth medium;
isolating single cell clones from the resulting polyclonal edited, selected polyclonal cell population;
amplifying at least one of the isolated single cell clones;
Assessing Genome Editing in at least one amplified single cell clone; and
obtaining a modified lepidopteran cell comprising a newly-introduced genome editing function resulting in a modified cellular phenotype.

25. A lepidopteran insect cell produced by the method of claim 24, wherein the newly-introduced genome editing function comprises reducing FDL function enough to reduce the cells ability to synthesize insect-type, paucimannosidic N-glycans (M3Gn2+/−Fuc) to less than 10% of total, as determined by MALDI-TOF-MS profiling of glycan structures.

26. A lepidopteran insect cell wherein FDL function is reduced enough to reduce the cells ability to synthesize insect-type, paucimannosidic N-glycans (M3Gn2+/−Fuc) to less than 10% of total, as determined by MALDI-TOF-MS profiling of glycan structures.

27. A method for obtaining a modified lepidopteran cell comprising a newly-introduced genome editing function resulting in a modified cellular phenotype, the method comprising:
transfecting a lepidopteran insect cell with the DNA vector of claim 18, wherein the vector further comprises SEQ ID NO:2 inserted into the targeting sequence cloning site and operably linked to a second transcriptional control element;
incubating the transfected cells in a selective growth medium;
isolating single cell clones from the resulting polyclonal edited, selected polyclonal cell population;
amplifying at least one of the isolated single cell clones;
Assessing Genome Editing in at least one amplified single cell clone; and
obtaining a lepidopteran cell comprising a modified genome editing function and a modified cellular phenotype.

28. A kit comprising the DNA vector of claim 1 comprising a lepidopteran insect U6 promoter; and cells derived from a lepidopteran insect.

29. The kit of claim 28, wherein the U6 promoter comprises SEQ ID NO: 47 or SEQ ID NO:51; and wherein the lepidopteran insect cells are derived from S. frugiperda, Trichoplusia ni, or Bombyx mori.

30. The kit of claim 29, wherein the lepidopteran insect cells comprise Sf-RVN cells.

31. The kit of claim 28, wherein the U6 promoter comprises SEQ ID NO: 51; and wherein the lepidopteran insect cells are derived from Trichoplusia ni.

32. The kit of claim 28, wherein the U6 promoter comprises SEQ ID NO: 53, SEQ ID NO: 54, or SEQ ID NO: 55; and wherein the lepidopteran insect cells are derived from Trichoplusia ni.

33. The kit of claim 28, wherein the U6 promoter comprises SEQ ID NO: 47, SEQ ID NO: 48, or SEQ ID NO: 51; and wherein the lepidopteran insect cells are derived from Bombyx mori.


SEQUENCE LISTING (55 sequences)

SEQ ID NO: 1 - DNA, 18 bp, Drosophila melanogaster
gcgccatattcatcctga

SEQ ID NO: 2 - DNA, 18 bp, Spodoptera frugiperda
ggcagtgcgatgaagtgg

SEQ ID NO: 3 - DNA, 18 bp, Spodoptera frugiperda
gccgcggcgctgctgtac

SEQ ID NO: 4 - DNA, 18 bp, Spodoptera frugiperda
gaagtgtcggaacgttgc

SEQ ID NO: 5 - DNA, 18 bp, Trichoplusia ni
gaagtgtccgagcgctgc

SEQ ID NO: 6 - DNA, 18 bp, Bombyx mori
gcgagaggtatcaagcat

SEQ ID NO: 7 - DNA, 18 bp, Bombyx mori
gctctggccacagccgac

SEQ ID NO: 8 - DNA, 18 bp, Bombyx mori
ggcctgtcagcctcgcat

SEQ ID NO: 9 - DNA, 18 bp, Artificial Sequence
  Synthetic
gggcgaggagctgttcac

SEQ ID NO: 10 - DNA, 48 bp, Artificial Sequence
  Synthetic
gatcccactagtgtcgacaccagtctctaattttttttttcaaaaaaa

SEQ ID NO: 11 - DNA, 48 bp, Artificial Sequence
  Synthetic
ctagccactagtgtcgacaccagtctctaattttttttttcaaaaaaa

SEQ ID NO: 12 - DNA, 48 bp, Artificial Sequence
  Synthetic
tcgaccactagtgtcgacaccagtctctaattttttttttcaaaaaaa

SEQ ID NO: 13 - DNA, 48 bp, Artificial Sequence
  Synthetic
agctccactagtgtcgacaccagtctctaattttttttttcaaaaaaa

SEQ ID NO: 14 - DNA, 61 bp, Artificial Sequence
  Synthetic
cgaagagtaaccgttgctaggagagaccgtggctgaatgagactggtgtcgacactagtgg

SEQ ID NO: 15 - DNA, 28 bp, Artificial Sequence
  Synthetic
cgaagagtaaccgttgctaggagagacc

SEQ ID NO: 16 - DNA, 25 bp, Artificial Sequence
  Synthetic
gtggctgaatgagactggtgtcgac

SEQ ID NO: 17 - DNA, 27 bp, Spodoptera frugiperda
gcttcacgattttgcgtgtcatccttg

SEQ ID NO: 18 - DNA, 26 bp, Spodoptera frugiperda
gggccatgctaatcttctctgtatcg

SEQ ID NO: 19 - DNA, 20 bp, Drosophila melanogaster
acaggcctggtggtggtgtc

SEQ ID NO: 20 - DNA, 27 bp, Drosophila melanogaster
aaagttaagatccccggatttgagcac

SEQ ID NO: 21 - DNA, 25 bp, Spodoptera frugiperda
ggcagtttctaaccgcttacttttg

SEQ ID NO: 22 - DNA, 23 bp, Spodoptera frugiperda
cttactcgtagagagcgtgcagc

SEQ ID NO: 23 - DNA, 22 bp, Spodoptera frugiperda
cgcggacttctccttgacacag

SEQ ID NO: 24 - DNA, 20 bp, Spodoptera frugiperda
cgaacccgcagtccaggtac

SEQ ID NO: 25 - DNA, 17 bp, Trichoplusia ni
atgaagtggtggggcga

SEQ ID NO: 26 - DNA, 19 bp, Trichoplusia ni
gccacagctgtgtcgagtc

SEQ ID NO: 27 - DNA, 21 bp, Bombyx mori
cttttatttatcgattcgggc

SEQ ID NO: 28 - DNA, 20 bp, Bombyx mori
gaatgcgctgtgatgtctac

SEQ ID NO: 29 - DNA, 140 bp, Bombyx mori
gtcgagtgttgttgtaaatcacgctttcaatagtttagtttttttaggtatatatacaaaatatcgtgctctacaagtgtacttgcttcggcagtacatatactaaaattggaacgatacagagaagattagcatggccc

SEQ ID NO: 30 - DNA, 140 bp, Spodoptera frugiperda
cgggagtaactatgactctcttaaggtagccaaatgcctcgtcatctaattagtgacgcgcatgaatggattaacgagattccctcttcggcagtacatatactaaaattggaacgatacagagaagattagcatggccc

SEQ ID NO: 31 - DNA, 140 bp, Spodoptera frugiperda
aatgtatgggattctacatcgcgctatgaaagttttcattgtgtttgtgagcggtacaataattttgccttagcaagtgtacttgcttcggcagtacatatactaaaattggaacgatacagagaagattagcatggccc

SEQ ID NO: 32 - DNA, 140 bp, Spodoptera frugiperda
taacatgaaactctaaatcgcgatatcaacatttttgttgtttggtgcctaatatacaaaaattcgtgctcgaccaccgtacttgcttcggcagtacatatactaaaattggaacgatacagagaagattagcatggccc

SEQ ID NO: 33 - DNA, 140 bp, Spodoptera frugiperda
aatgtatgggattgtacatcgcgctattaaagttttcattgtgtttgtgagcggtacaataattttgccttagcaagtgtacttgcttcggcagtacatatactaaaattggaacgatacagagaagattagcatggccc

SEQ ID NO: 34 - DNA, 140 bp, Spodoptera frugiperda
caaatgtccgaaactgcggttcctctcgtactgagcagtattactatcgcaacgacaagccatcagtagggtaaaaccggttcggcttcggcagtacatatactaaaattggaacgatacagagaagattagcatggccc

SEQ ID NO: 35 - DNA, 140 bp, Spodoptera frugiperda
aatgtatgagattctacatcgcgctatcaaagtttttattgtgtttgtgagcggtacaataattttgccatagcaagtgtacttgcttcggcagtacatatactaaaattggaacgatacagagaagattagcatggccc

SEQ ID NO: 36 - DNA, 140 bp, Spodoptera frugiperda; identical to SEQ ID NO: 32

SEQ ID NO: 37 - DNA, 140 bp, Trichoplusia ni
gtgtccatgaggattcccccgcctaaaaaaaaaaaaaaaaaaaaaaaaaaaaggttaggttaggttggggtgcagggtgtacttgcttcggcagtacatatactaaaattggaacgatacagagaagattagcatggccc

SEQ ID NO: 38 - DNA, 140 bp, Trichoplusia ni
cctttcaaatcctgaatcgcacaatcaaagttttcacttgttatcggcatccattctgtatattcgacccctaacattgtacttgcttcggcagtacatatactaaaattggaacgatacagagaagattagcatggccc

SEQ ID NO: 39 - DNA, 140 bp, Trichoplusia ni
ccttcaaaatcctgaatcgcgcaatcgaaatgcttttaattcatcagtatacgaacgtctacttcgacccctaacatcgtacttgcttcggcagtacatatactaaaattggaacgatacagagaagattagcatggccc

SEQ ID NO: 40 - DNA, 140 bp, Trichoplusia ni
tgccaaaaattctgaatcgcacaatcaaagttttcaactgttatcggcatccattctgtatattcgacccctaacattgtacttgcttcggcagtacatatactaaaattggaacgatacagagaagattagcatggccc

SEQ ID NO: 41 - DNA, 140 bp, Trichoplusia ni
atgtatagagttctgaatcgcgcaatcaaagttgtccaattttataggtacagattaagtttttggcgctcatatttcgtacttgcttcggcagtacatatactaaaattggaacgatacagagaagattagcatggccc

SEQ ID NO: 42 - DNA, 140 bp, Trichoplusia ni
ccgggagggcgggtgtccatgaggattcccccgcctaaaaaaaaaaaaaaaaaaaaaaaaaaaaaaaaaaaaaaggttgtacttgcttcggcagtacatatactaaaattggaacgatacagagaagattagcatggccc

SEQ ID NO: 43 - DNA, 140 bp, Trichoplusia ni
acgtgggcattcctgtatcacgcaatcaaagttcgctgagattgtaggtatggtttctattattggcgcatttcaactgtactcgcttcggcagtacatatactaaaattggaacgatacagagaagattagcatggccc

SEQ ID NO: 44 - DNA, 140 bp, Trichoplusia ni
ccttaacagtatttaaattgtatttctaaatatggactctggttcgaaataaagatattattattattattattattcgtacttgcttcggcagtacatatactaaaattggaacgatacagagaagattagcatggccc

SEQ ID NO: 45 - DNA, 82 bp, Artificial Sequence
  SyntheticsgRNA
gttttagagctagaaatagcaagttaaaataaggctagtccgttatcaacttgaaaaagtggcaccgagtcggtgctttttt

SEQ ID NO: 46 - DNA, 9596 bp, Artificial Sequence
  Synthetic recombinant plasmid
tcgatgtctttgtgatgcgcgcgacatttttgtaggttattgataaaatgaacggatacgttgcccgacattatcattaaatccttggcgtagaatttgtcgggtccattgtccgtgtgcgctagcatgcccgtaacggacctcgtacttttggcttcaaaggttttgcgcacagacaaaatgtgccacacttgcagctctgcatgtgtgcgcgttaccacaaatcccaacggcgcagtgtacttgttgtatgcaaataaatctcgataaaggcgcggcgcgcgaatgcagctgatcacgtacgctcctcgtgttccgttcaaggacggtgttatcgacctcagattaatgtttatcggccgactgttttcgtatccgctcaccaaacgcgtttttgcattaacattgtatgtcggcggatgttctatatctaatttgaataaataaacgataaccgcgttggttttagagggcataataaaagaaatattgttatcgtgttcgccattagggcagtataaattgacgttcatgttggatattgtttcagttgcaagttgacactggcggcgacaagatcgtgaacaaccaagtgacaacatggactacaaggaccacgacggcgattacaaggatcacgacatcgactacaaggacgatgacgacaagatggcccccaagaagaagcgcaaagtcggtatccacggtgtccccgctgctgacaagaagtactccatcggcctggacatcggcaccaactccgtgggctgggctgtgatcaccgacgagtacaaggtgccctccaagaagttcaaggtcctgggcaacaccgaccgtcactccatcaagaagaacctgatcggcgctctgctgttcgactccggcgagactgctgaggctacccgtctgaagcgtaccgctcgtcgtcgttacacccgtcgcaagaaccgtatctgctacctgcaagagatcttctccaacgagatggctaaggtggacgacagcttcttccaccgtctggaagagtccttcctggtggaagaggacaagaagcacgagcgtcaccccatcttcggcaacatcgtggacgaggtggcctaccacgagaagtaccccaccatctaccacctccgcaagaagctggtcgactccaccgacaaggctgacctgcgtctgatctacctggctctggctcacatgatcaagttccgtggtcacttcctgatcgagggcgacctgaaccccgacaactccgacgtggacaagctgttcatccagctggtgcagacctacaaccagctgttcgaggaaaaccccatcaacgcttccggtgtcgacgctaaggctatcctgtccgctcgtctgtccaagtcccgtcgtctggaaaacttgatcgctcagctgcccggcgagaagaagaacggcctgttcggcaacctgatcgctctgtccctgggcctgacccccaacttcaagtccaacttcgacctggctgaggacgctaagctccagctgtccaaggacacctacgacgatgacctggacaacctgctggctcagatcggcgaccagtacgctgacctgttcctggctgctaagaacctgtccgacgctatcctgctgtccgacatcctgcgtgtgaacaccgagatcaccaaggctcctctgtccgcttctatgatcaagcgttacgacgagcaccaccaggacctgaccctgctgaaggctctcgtgcgtcagcagctgcctgagaagtacaaggaaatcttcttcgaccagtccaagaacggctacgctggttacatcgacggtggtgcttcccaagaggaattctacaagttcatcaagcccatcctcgagaagatggacggcaccgaggaactgctggtcaagctgaaccgcgaggacctgctgcgcaagcagcgcaccttcgacaacggttccatcccccaccagatccacctgggcgagttgcacgctatcttgcgtcgtcaagaggacttctacccattcctgaaggacaaccgcgagaagatcgaaaagatcctgaccttccgtatcccctactacgtgggtcccctggctcgtggcaactcccgtttcgcttggatgacccgcaagtccgaggaaaccatcaccccctggaacttcgaagaggtggtggacaagggcgcttccgctcagtccttcatcgagcgtatgactaacttcgacaagaacctgcccaacgagaaggtgctgcccaagcactccctgctgtacgagtacttcaccgtgtacaacgagctgaccaaagttaaatacgtgaccgagggaatgcgcaagcccgctttcctgtccggcgagcaaaagaaggctatcgtcgacctgctgttcaagaccaaccgcaaagtgaccgtgaagcagctgaaggaagattacttcaagaagatcgagtgcttcgacagcgtcgagatctccggcgtcgaggaccgtttcaacgcctccctgggcacttaccacgacctgctcaagatcatcaaggacaaggatttcttggacaacgaagagaacgaggacatcttggaggacatcgtgctgaccctgaccctcttcgaggacagagagatgatcgaggaacgcctcaagacctacgctcacttgttcgacgacaaagtgatgaagcaactcaagcgtcgccgctacaccggctggggtcgtctgtctcgcaagctgatcaacggtatccgtgacaagcagtccggcaagactatcctggacttcctgaagtccgacggtttcgctaaccgtaacttcatgcagctgatccacgacgactccctgactttcaaggaggacatccaaaaggctcaggtgtccggccagggcgactctctgcacgagcacatcgctaacctggctggttcccccgctatcaagaagggtatcctgcagaccgtcaaggtggtcgacgaactggtcaaagtcatgggtcgtcacaagcccgagaacatcgtcatcgagatggcccgcgagaaccagaccacccagaagggtcaaaagaactcccgcgagcgcatgaagcgtatcgaagaaggcatcaaggaactgggttcccagatcctcaaggaacaccccgtcgagaacacccagctgcagaacgagaagctgtacctgtactacctccagaacggtcgcgatatgtacgtggaccaagagctggacatcaaccgtctgtccgactacgatgtcgaccacatcgtgccccagtctttcttgaaggacgactcgatcgacaacaaggtgctgactcgttccgataagaaccgtggaaagtccgacaacgtcccctccgaagaggtcgtgaagaagatgaagaactactggcgtcagctgctcaacgccaagctcatcacccagaggaagttcgacaacttgaccaaggctgagcgtggtggcctgtccgaactggacaaggccggtttcatcaagaggcagctggtggaaacccgtcagatcactaagcacgtggcccagatcttggactcccgtatgaacactaagtacgacgagaacgacaagttgatccgcgaagtgaaagtgatcaccctcaagtctaagctggtgtccgacttccgcaaggacttccagttctacaaagtgcgcgagatcaacaactaccaccacgcccacgacgcttacctgaacgctgtcgtgggcaccgccctcatcaagaagtaccctaagctcgagtccgagttcgtgtacggcgactacaaggtgtacgacgtgcgcaagatgatcgctaagtccgagcaagaaatcggcaaggctaccgccaagtacttcttctactccaacatcatgaacttcttcaagactgagatcaccctggccaacggcgagatccgcaagcgtcctctgatcgagactaacggcgaaactggcgagatcgtgtgggacaagggtcgtgacttcgctaccgtcagaaaggtgctgtccatgccccaagtgaacatcgttaagaagaccgaggtccagaccggtggtttctccaaggaatccatcctgcctaagaggaactccgataagctgatcgctaggaagaaggactgggaccctaagaagtacggcggtttcgactcccccaccgtggcttactctgtgctggtggtcgctaaggtcgagaagggaaagtctaagaagctcaagtccgtcaaggaattgctgggcatcaccatcatggaacgctccagcttcgagaagaaccctatcgacttcctcgaggctaagggctacaaggaagtcaagaaggacctcatcatcaagctccccaagtacagcctgttcgagctggaaaacggtcgcaagcgtatgctggcttccgctggcgaactgcagaagggcaacgaactggctctgccctctaaatacgtcaacttcctgtacctggcttcccactacgaaaagctgaagggctcccccgaggataacgaacaaaagcaactgttcgtcgagcagcacaagcactacctggacgagatcatcgagcagatctccgagttctccaagcgtgtgatcctggctgacgctaacctcgataaggtgctctccgcttacaacaagcaccgcgacaagcctatccgcgagcaggctgagaacatcatccacctgttcaccctgactaacctgggtgctcccgctgctttcaagtacttcgacaccaccatcgaccgcaagcgctacacctccaccaaggaagtgctcgacgctaccctgatccaccagtccatcaccggcctgtacgagactcgtatcgacctgtcccagctcggtggcgacaagcgtccagctgctaccaagaaggctggccaggctaagaagaagtaatgtaaacgccacaattgtgtttgttgcaaataaacccatgattatttgattaaaattgttgttttctttgttcatagacaatagtgtgttttgcctaaacggtttgggagatctaagcttcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaattggatcccgggccggctaattcgttcgacttgcagcctgaaatacggcacgagtaggaaaagccgagtcaaatgccgaatgcagagtctcattacagcacaatcaactcaagaaaaactcgacacttttttaccatttgcacttaaatccttttttattcgttatgtatactttttttggtccctaaccaaaacaaaaccaaactctcttagtcgtgcctctatatttaaaactatcaatttattatagtcaataaatcgaactgtgttttcaacaaacgaacaataggacactttgattctaaaggaaattttgaaaatcttaagcagagggttcttaagaccatttgccaattcttataattctcaactgctctttcctgatgttgatcatttatataggtatgttttcctcaatacttcggaagagcgatatcaagcttggtacccaagctcttccgttttagagctagaaatagcaagttaaaataaggctagtccgttatcaacttgaaaaagtggcaccgagtcggtgcttttttctgcagactagtgcggccgcaaatgtttgggccgcgtaaaacacaatcaagtatgagtcataagctgatgtcatgttttgcacacggctcataaccgaactggctttacgagtagaattctacttgtaacgcacgatcgagtggatgatggtcatttgtttttcaaatcgagatgatgtcatgttttgcacacgggctcataaactgctttacgagtagaattctacgtgtaacgcacgatcgattgatgagtcatttgttttgcaatatgatatcatacaatatgactcatttgtttttcaaaaccgaacttgatttacgggtagaattctactcgtaaagcacaatcaaaaagatgatgtcatttgtttttcaaaactgaactctcggctttacgagtagaattctacgtgtaaaacacaatcaagaaatgatgtcatttgttataaaaataaaagctgatgtcatgttttgcacatggctcataactaaactcgctttacgggtagaattctacgtcgatgtctttgtgatgcgccgacatttttgtaggttattgataaaatgaacggatacagttgcccgacattatcattaaatccttggcgtagaatttgtcgggtccattgtccgtgtgcgctagcatgcccgctaacggacctcgtacttttggcttcaaaggttttgcgcacagacaaaatgtgccacacttgcagctctgcatgtgtgcgcgttaccacaaatcccaacggcgcagtgtacttgttgtatgcaaataaatctcgataaaggcgcggcgcgcgaatgcagctgatcacgtacgctcctcgtgttccgttcaaggacggtgttatcgacctcagattaatgtttatcggccgactgttttcgtatccgctcaccaaacgcgtttttgcattaacattgtatgtcggcggatgttctatatctaatttgaataaataaacgataaccgcgttggttttagagggcataataaaagaaatattgttatcgtgttcgccattagggcagtataaattgacgttcatgttggatattgtttcagttgcaagttgacactggcggcgacaagatcgtgaacaaccaagtgacgcggatctagatctcgagcggccgcaccatgaccgagtacaagcccaccgtgcgtctggctacccgtgacgatgtgcctcgtgctgtgcgtaccctggctgctgctttcgctgactaccccgctacccgtcacaccgtggatcccgaccgtcacatcgagcgtgtgaccgagctgcaagagctgttcctgacccgtgtgggcctggacatcggcaaagtgtgggtggccgacgacggtgctgctgtggctgtgtggaccacccctgagtccgtggaagctggtgctgtgttcgctgagatcggtccccgtatggctgagctgtccggttcccgtctggctgctcagcagcagatggaaggcctgctggctccccaccgtcctaaggaacctgcctggttcctggctaccgtgggcgtgtcacctgaccaccagggaaagggactgggttccgctgtggtgctgcctggtgtcgaggctgctgaacgtgctggtgtccccgctttcctggaaacctccgctccccgtaacctgcccttctacgagcgtctgggtttcaccgtgaccgctgacgtggaagtgcccgagggtcctcgtacctggtgcatgactcgcaagcccggtgcttaagtttcgatgtaaacgccacaattgtgtttgttgcaaataaacccatgattatttgattaaaattgttgttttctttgttcatagacaatagtgtgttttgcctaaacggtttaaacggcgcgcccctaggtac

SEQ ID NO: 47 - DNA, 335 bp, Spodoptera frugiperda
  misc_feature (1)..(335): Spodoptera frugiperda [SfU6]
ctagaaacggattacgagttaactagcgccatctgttgttgtgtaagtaacaacactgatatacttgtgtggaatagttccgacagaatttgtagatggcgctgtaataaaaatattatttaaaaacatgtatttttcacaattttatatattattgtaagatatttcgtgatattttataataaaaaatacattaatagtaaatattgtaattaaaaaaaggtttcaccttatttcattaaagattttaagaaatataacatgaaactctaaatcgcgatatcaacatttttgttgtttggtgcctaatatacaaaaattcgtgctcgaccacc

SEQ ID NO: 48 - DNA, 467 bp, Bombyx mori
  misc_feature (1)..(467): Bombyx mori  [BmU6-2]
aggttatgtagtacacattgttgtaaatcactgaattgttttagatgattttaacaattagtacttattaatattaaataagtacataccttgagaatttaaaaatcgtcaactataagccatacgaatttaagcttggtacttggcttatagataaggacagaataagaattgttaacgtgtaagacaaggtcagatagtcatagtgattttgtcaaagtaataacagatggcgctgtacaaaccataactgttttcatttgtttttatggattttattacaaattctaaaggttttattgttattatttaatttcgttttaattatattatatatctttaatagaatatgttaagagtttttgctctttttgaataatctttgtaaagtcgagtgttgttgtaaatcacgctttcaatagtttagtttttttaggtatatatacaaaatatcgtgctctacaagt

SEQ ID NO: 49 - DNA, 764 bp, Trichoplusia ni
  misc_feature (1)..(764): Trichoplusia ni  [TnU6-2]
gatctgctttatgtacttcaatgtaacaatttcaatttttttttgttctaaaaatgttcacaagtactacagtgaatcaccatccaggataaaagaaattaacttcatttacacccattatactatgaaacaatgtacgtaggtaagtagcttctctaacacattattcttcaaacaaactaactcaatctccaattggataaaaacctttttgctacaacgcgttcaaaacaaagagtgttttatttttaaacggttaccactcaattcgtttttgccgttagcattaaaaaacaaacagttttccattagcttaatcgttgcttcataaaagggccttttattggatattagaacactgtcccatatagctatatgatatggggttgccataatttcacggtgaccgcgtggcaggtgaattaagtcaaccataaaactcatgtggtatttttttctttgaaaagttttttaattaagaaatttgtttcaacacttctttttttttgttttgatgtttgatattttaatggtaaagcagtatgtttttttctataaatgtttgaataaaaaatattaatagttgattacactacacagacacattttacaaatacctatagtaagtgcgatgcattccccctttacctttctattgaatggaggcagctccaagttgttgtcaccctttcaaatcctgaatcgcacaatcaaagttttcacttgttatcggcatccattctgtatattcgacccctaacatt

SEQ ID NO: 50 - DNA, 577 bp, Trichoplusia ni
  misc_feature (1)..(577): Trichoplusia ni  [TnU6-3]
gatctcatgagagtcagtgattcaactcaactacaaccttactatattactttattttcagaatactaaatactttaatcgcttggatgtcgcaagtcgttttttttggagtttaagcttacctatatgaagttttatgagaattcgttaagcattttagtattaaaaagttgatggacacaattacttttataattatttctatgaattaagttttagttctcattgcaaagtattttatttctattttatgacttacttgtaaattaaggtacgtaagtatctaaactattttatagagcgtacttttaaaaacttttaataataaacgcattttagtctacattaatattttgcataaaatttaaatcatagtaaagtaagtttggaccttaaataaaattcaaaaaataaaatcaaaccgtgcggagcgaagacgggacagacaccttggcgcattttcgtctgtctctatcgacccttcacgcttttttgtcacccttcaaaatcctgaatcgcgcaatcgaaatgcttttaattcatcagtatacgaacgtctacttcgacccctaacatc

SEQ ID NO: 51 - DNA, 837 bp, Trichoplusia ni
  misc_feature (1)..(837): Trichoplusia ni [TnU6-4]
ctagtggacgtttcgtcgttatgtgctcagtatatcacagctattatcattgtttatatctcgtgttcttactaatgactgatgaggagcttatggcggtcattacaaacgttttcggggaaaaatcccatgaatacgtgctcagaattcatagcctgcttaactaactcctttcgtggcctataaggcagttagatatgtacctagtgtttttttacgggtaataggatgagcaaagtctattttacaactaagagttttggcggagaactaaagcagtagaaaagtaggtacataaaatcacattaggtattacagcgccatctatacattacatgtgaaactttttgcagaccaagctaacaagaagtttatgagtttgtctctttgctatttagtgtataaaggagattgtagatggcgttgctaaagaccgtcagaatattatataaaagtaaaatatactagaaatatggaaccagtataccttcttattaaataaaacactattagtactaattataaataacaatttctatgttttttcttgcacttgtgcaatctaagttacagctagtttcatgtcgttttactagatggcgcttcgaacacactatgcgtaaaaaataaaaataagtcatatttgctgttacaaaatcaaattaaataacaacgaaaatacaaaaactgttttcaagtattaaaagaataaaaaaggagaacatcttttttctggatatatggaactagaagcactaatgccaaaaattctgaatcgcacaatcaaagttttcaactgttatcggcatccattctgtatattcgacccctaacatt

SEQ ID NO: 52 - DNA, 610 bp, Trichoplusia ni
  misc_feature (1)..(610): Trichoplusia ni  [TnU6-5]
ctagacaaaactgtgatttttttgcagatgtgaaggtgattacagtttgattgtatgtgagatgattgcaggttttggtttttatattttactagctgttgcccgcgacttcgtctgcgtggttagaagatataagttatgactttttcttcgctcctattggtcgcagcgtgatgatatatagcctaaaacctctctcgatgaatggtccattcaacactaaaatattttttcaatttgaaccagtagttctgttctgaaaaatacaaaaaataaattaaatataatgctatttgcgcactcatccacacaaaaaattgcgcaatcagaaaataatgcgacatcatttcgctcgttgttgtggaaaaataaaagtttttgtaaaattattaaaaacaagttttaggtcttatctgattatattaaccatgaaatctacaaattcagtaatttaaggaaaaaaaatacttttttctttgtaggcatggcataataattcatttggtgttaacattcctcaatagatggcgcaatgtatagagttctgaatcgcgcaatcaaagttgtccaattttataggtacagattaagtttttggcgctcatatttc

SEQ ID NO: 53 - DNA, 1000 bp, Trichoplusia ni
  misc_feature (1)..(1000): Trichoplusia ni  [TnU6-9]
caataaattaatgcctaaagtgctttcgtcgtacattttgatgttagaaaacactcatattagggaatacttttttacatatggcaactgtcttaaaaaaagtgtaaaagcaagaaatgaatgtgcgatttgttattttttatgtttatattgataaataaataataaaaatgtgtctcgtatgcgtggaaatggacattaagggaaaaaaaaaaacaattggtggaattatttatatacagaaccagatgatttaagtgtttataatcagtaaaaccaagcgtggattatagatttttatatttactaaacaaaattgttataaaaataagtaatatttggaaaataaagtaaagtgctccggttaacaacattgtacattttgttttgatagtgcaattataaattctgtaatggcaaatcattggaaatatcttgtaccatagaaataaattatgtatttaaaacgaatgtctattttattcagctgagggcgtaatctggccttcgttgtcgtgataagagtcgcatcagaattaaatataaatcaattgttaaaacttgtttgtttttacctagatttaacttaataatgcatttattaaaaatagcaaaacaacagccatccgagtttctgctgtactctcaaataggtaactggctaagatgtgattgagaacagcgccatctacattcttcggatttaaactcttggtgcgctagttcgcatgtgttttcgttagttcatacctgtgacacagatgtcgctagtgtggcaagattaaaacaatttgcttattttctcttataaatcaattaaatgagagagaaatgaaactttctgacccaaagattgaataattaattattgttttacaaatatatgtaagttttttttttaaatgtaagtaattcttaagttattacagataattttaactctcgcaattgatgtgtttggtccaataagctattgaatcgtatttggtgcaccacaagt

SEQ ID NO: 54 - DNA, 1000 bp, Trichoplusia ni
  misc_feature (1)..(1000): Trichoplusia ni  [TnU6-10]
gataatgaaaacttttgctataggactcatgccttatctgataataagaacaatttaatctacaaaacaaaatcaatcctgtaagtaaagaaaaatagtttttcaagattgtcaatagatggcgctgttacaaccttccttacatttagagatgcagtttcaaattaaaagaaaatacaatttaccgtgtttaatagcaaataatacaaatatttgttgtttcgaactatttaatctattgataaattgtcttcagtgatatcagataagatatcgtttaaagtaggtacacaatcttgtaaatatcattatctaacagatgttttaaattgttgctatatttatgatgaatcatattatgatcgccgatagttgctaacacacccgcaaccccattccgtacggggtttcttacgatttaaaaattcagtacaggacggaacactcaaaatgctttaaagaatacgagtaatgacaaatttaatccaggtgccattcattcaatggccattgcaaatagagaacctgattgaggcccagcccaggcttatgatacttatacttaccttcaatacgcaataatagcagtttgtgtacacctaaaagacaaattctttttttataaaacgctttttttcgaaatctcattgaaacattgctttgggacgcaattgatagtacaccatataaaaacattggtccatcacagaacattgtgaatagaaatagctttgtgtgttgacatattttaaataaatatttgtatgttttagttgataattaaaatttacaactatcctgaatggttagttttcatctctgcaatgcattgtagcgaccgtggctgcataaaaaaaatatttagtttttttatattgatcgatagatggcgctgttacaagcttccgaacatcagtcgttggagtatggcgttctgaatcgcgcaatcaaagttatcacgttttgtaggtatagttctaatatttagcgggttacattc

SEQ ID NO: 55 - DNA, 999 bp, Trichoplusia ni
  misc_feature (1)..(999): Trichoplusia ni  [TnU6-11]
taagtaaacaaaaatatcattttattttctttaaagttaaattttattaatttacaagaagtactgttttcataaaatttttaagtttgggtagatttttctattattatttgaatttatttgtaatgaggctgatcatttattaaccttcagagatctttataataatgggtgtattttcaactaaataaaatacaaaaagaaaaatttaacaaaatagaaatgagaaaaggtatttttattatttttcacttaaaataaaacttgaatattgttacataattttggaattgctaaaataaaaacaaaagtcctatttttaatatgtaagttaaggtctctgctaggtattattgaaatgttcactctcataaacatagtttttaaataaatacatgtaggtaaaatatacgaatttatatattaagtaaaaaatacttttgtaaatgtcttaaactgccaagggctattgattttaggaaaattagttccaataattcaactaaaattattaaaactaaagtatttttacaatttagaaaaattaaaataaaataattttagttaaaaacggattccgtccataatttatttttagataaacatttgtaaatatttttcgttttttttttatatgaaatgcatttattaattttatttaattagccataataatgtgacagagttaatctggattctgattttatgaatttattaaatcattttaaaaagttatttgtattttgattccatctcttaattactcatttagtaatttattaatattaaatcgtaattatttaactacactgcaaaattttaataccgcatccatgtgttctacctttactcaatgttgtggtaattactagattcgtcgatagatggcgctgtgactgctcctcatacattgtaagcgttgtctgtgaaatcctaaatcgcgcaatcaaaattgtcacgttttgtaggtatagtactaatatttagcatattacattc